(19) United States
(12) Patent Application Publication
(10) Pub. No.: US 2026/0216199 A1
(43) Pub. Date: Jul. 30, 2026

(54) RAF INHIBITOR AND KRAS G12C INHIBITOR COMBINATION THERAPY

(71) Applicant: Erasca, Inc., San Diego, CA (US)

(72) Inventors: Wei LIN, San Diego, CA (US); Jonathan LIM, San Diego, CA (US); Shannon MORRIS, San Diego, CA (US); Robert Field SHOEMAKER, San Diego, CA (US); Jae Hyun BAE, San Diego, CA (US); Zachary Miles PARTON, San Diego, CA (US)

(21) Appl. No.: 19/145,652

(22) PCT Filed: Jan. 5, 2024
(86) PCT No.: PCT/US2024/010524

§ 371 Date: Jul. 3, 2025

Related U.S. Application Data
(60) Provisional application No. 63/478,888, filed on Jan. 6, 2023.
(60) Provisional application No. 63/505,643, filed on Jun. 1, 2023.

(51) Int. Cl. A61K 31/5377; A61K 31/519; A61K 31/542; A61K 39/395; A61P 35/00

(52) U.S. Cl. CPC A61K 31/5377; A61K 31/519; A61K 31/542; A61K 39/3955; A61P 35/00

(57) ABSTRACT

A pharmaceutical combination including a Raf inhibitor which is Compound of formula (I) or a pharmaceutically acceptable salt thereof, (b) a KRAS G12C inhibitor, and/or (c) trametinib.

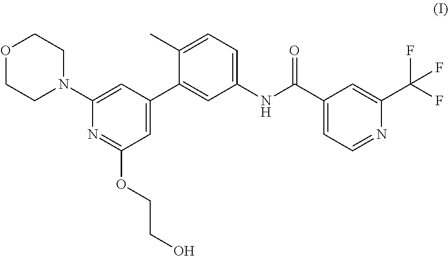 [SMILES: Cc1ccc(NC(=O)c2ccnc(C(F)(F)F)c2)cc1-c1cc(OCCO)nc(N2CCOCC2)c1]

### CROSS-REFERENCE

This application claims the benefit of U.S. Provisional Application Ser. No. 63/478,888 filed Jan. 6, 2023, and U.S. Provisional Application Ser. No. 63/505,643 filed Jun. 1, 2023, which are hereby incorporated by reference in their entirety.

### BACKGROUND

The RAS/RAF/MEK/ERK or MAPK pathway is a key signaling cascade that drives cell proliferation, differentiation, and survival. Dysregulation of this pathway underlies many instances of tumorigenesis. This pathway is activated by extracellular signals that in turn induces the small G protein RAS to exchange GDP for GTP. The activated RAS small guanidine triphosphatase (GTPase) promotes the activation of the RAF (also referred to as Raf herein) family proteins (ARAF, BRAF and CRAF, also known as RAF1). Activated RAF proteins lead to the phosphorylation and activation of MEK1/2 proteins, which subsequently phosphorylate and activate extracellular signal regulated kinases (ERKs). ERK1/2 proteins phosphorylate a variety of substrates, including multiple transcription factors, and regulate key cellular activities, including proliferation, differentiation, migration, survival, and angiogenesis.

Aberrant signaling or inappropriate activation of the MAPK pathway has been shown in multiple tumor types, including colorectal, lung and pancreatic cancer, and can occur through several distinct mechanisms, including activating mutations in RAS and BRAF (V-Raf Murine Sarcoma Viral Oncogene Homolog B 1). RAS which is a superfamily of GTPases includes KRAS (v-Ki-ras2 Kirsten rat sarcoma viral oncogene homolog), which is a regulated signaling protein that can be turned on (activated) by various single-point mutations, which are known as gain-of-function mutations. RAS mutations, particularly gain-of-function (GOF) mutations, have been detected in 9-30% of all cancers, with KRAS mutations having the highest prevalence (86%), followed by NRAS (11%), and, infrequently, HRAS (3%) (Cox A D, et al, Nat Rev Drug Discov 2014; 13(11):828-51). Activating KRAS mutations are also frequently found in melanoma (Fedorenko I V, et al, Br J Cancer 2015; 112(2):217-26), pancreatic cancer (di Magliano M P & Logsdon C D, Gastroenterology 2013; 144(6): 1220-9), colorectal cancer (Knickelbein K & Zhang L, Genes Dis 2015; 2(1):4-12) and ovarian cancer (Nakayama N, et al, Br J Cancer 2008; 99(12):2020-8).

### SUMMARY

Embodiments herein relate to a pharmaceutical combination comprising (a) a Raf inhibitor which is a Compound of formula (I)

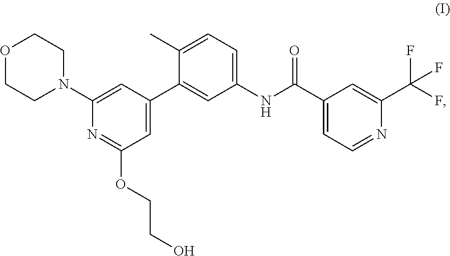 [SMILES: Cc1ccc(NC(=O)c2ccnc(C(F)(F)F)c2)cc1-c1cc(OCCO)nc(N2CCOCC2)c1]

or a pharmaceutically acceptable salt thereof,
and (b) a KRAS G12C inhibitor or a pharmaceutically acceptable salt thereof.

Embodiments herein further relate to a pharmaceutical combination comprising (a) a Raf inhibitor which is a Compound of formula (I)

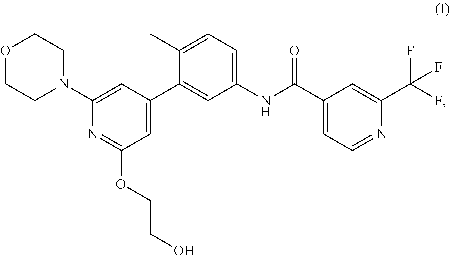 [SMILES: Cc1ccc(NC(=O)c2ccnc(C(F)(F)F)c2)cc1-c1cc(OCCO)nc(N2CCOCC2)c1]

or a pharmaceutically acceptable salt thereof, (b) a KRAS G12C inhibitor or a pharmaceutically acceptable salt thereof, and (c) trametinib or a pharmaceutically acceptable salt thereof.

Embodiments further relate to a pharmaceutical combination comprising (a) a Raf inhibitor which is a Compound of formula (I), as defined above, or a pharmaceutically acceptable salt thereof, and a KRAS G12C inhibitor or a pharmaceutically acceptable salt thereof for use in the treatment of a proliferative disease, in particular a cancer, uses of such combinations for the preparation of a medicament for the treatment of a proliferative disease, in particular a cancer; there are further provided methods of treating a proliferative disease, in particular a cancer, in a subject in need thereof comprising administering to said subject a jointly therapeutically effective amount of said combinations; there are further provided use of such combinations for the treatment of a proliferative disease, in particular a cancer; pharmaceutical compositions comprising such combinations and commercial packages thereto.

Embodiments further relate to a pharmaceutical combination comprising (a) a Raf inhibitor which is a Compound of formula (I), as defined above, or a pharmaceutically acceptable salt thereof, (b) a KRAS G12C inhibitor or a pharmaceutically acceptable salt thereof, and (c) trametinib or a pharmaceutically acceptable salt thereof for use in the treatment of a proliferative disease, in particular a cancer, uses of such combinations for the preparation of a medicament for the treatment of a proliferative disease, in particular a cancer; there are further provided methods of treating a proliferative disease, in particular a cancer, in a subject in need thereof comprising administering to said subject a jointly therapeutically effective amount of said combinations; there are further provided use of such combinations for the treatment of a proliferative disease, in particular a cancer; pharmaceutical compositions comprising such combinations and commercial packages thereto.

### DETAILED DESCRIPTION

In some embodiments, there are provided pharmaceutical combinations comprising (a) a Raf inhibitor which is a which is Compound of formula (I)

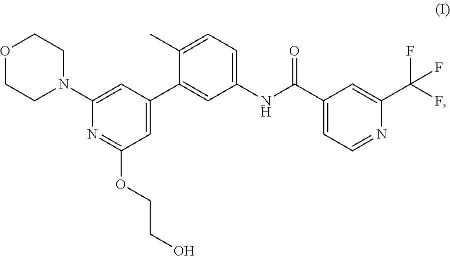 [SMILES: Cc1ccc(NC(=O)c2ccnc(C(F)(F)F)c2)cc1-c1cc(OCCO)nc(N2CCOCC2)c1]

or a pharmaceutically acceptable salt thereof,
and (b) a KRAS G12C inhibitor.

In some embodiments, there are provided pharmaceutical combinations comprising a Raf inhibitor which is Compound of formula (I)

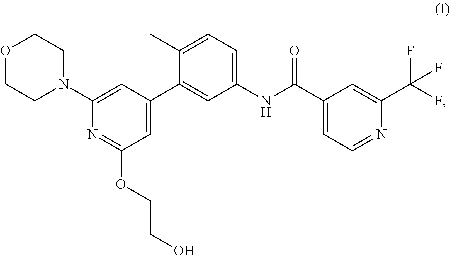 [SMILES: Cc1ccc(NC(=O)c2ccnc(C(F)(F)F)c2)cc1-c1cc(OCCO)nc(N2CCOCC2)c1]

or a pharmaceutically acceptable salt thereof,
(b) a KRAS G12C inhibitor,
and (c) trametinib.

In cell-based assays, Raf inhibitors Compound of formula (I) demonstrated anti-proliferative activity in cell lines that contain a variety of mutations that activate MAPK signaling. In vivo, treatment with Compound of formula (I) generated tumor regression in several KRAS-mutant models including the NSCLC-derived Calu-6 (KRAS Q61K) and NCI-H358 (KRAS G12C). Collectively, in vitro and in vivo MAPK-pathway suppression and anti-proliferative activity observed for Compound of formula (I) at well-tolerated doses suggest that Compound of formula (I) may have anti-tumor activity in patients with tumors harboring activating lesions in the MAPK pathway. Moreover, Compound of formula (I) are a Type 2 ATP-competitive inhibitor of both B-Raf and C-Raf that keeps the kinase pocket in an inactive conformation, thereby reducing the paradoxical activation seen with many B-Raf inhibitors, and blocking mutant Ras-driven signaling and cell proliferation. Compound of formula (I) exhibited efficacy in numerous MAPK-driven human cancer cell lines and in xenograft tumors representing model tumors harboring human lesions in KRAS, NRAS and BRAF oncogenes. Pharmaceutical combinations of the present embodiments further comprise a KRAS G12C inhibitor. Further pharmaceutical combinations of the present embodiments further comprise a KRAS G12C inhibitor and trametinib. The term “KRAS G12C inhibitor” is defined herein to refer to a compound which targets, decreases, or inhibits the KRAS mutation of glycine 12 to cysteine.

In some embodiments, suitable KRAS G12C inhibitors are selected from sotorasib, adagrasib,

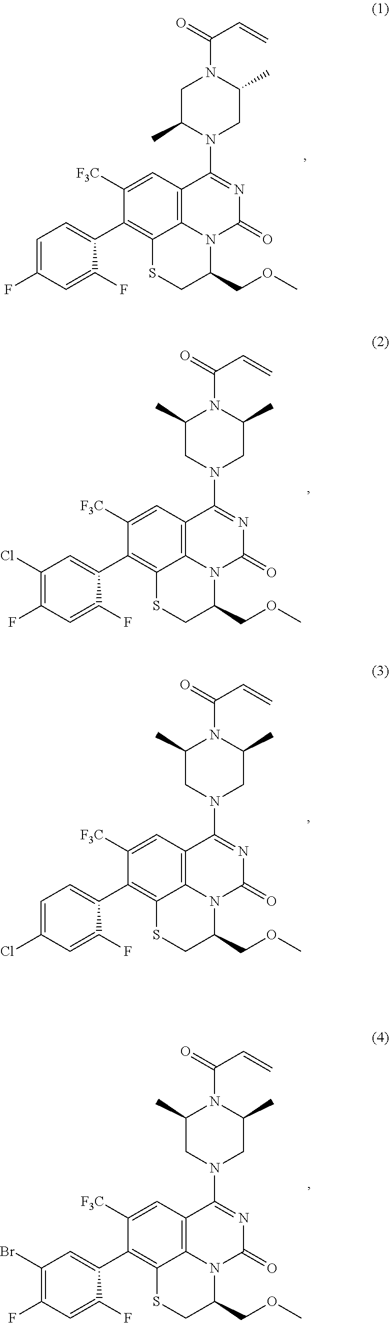

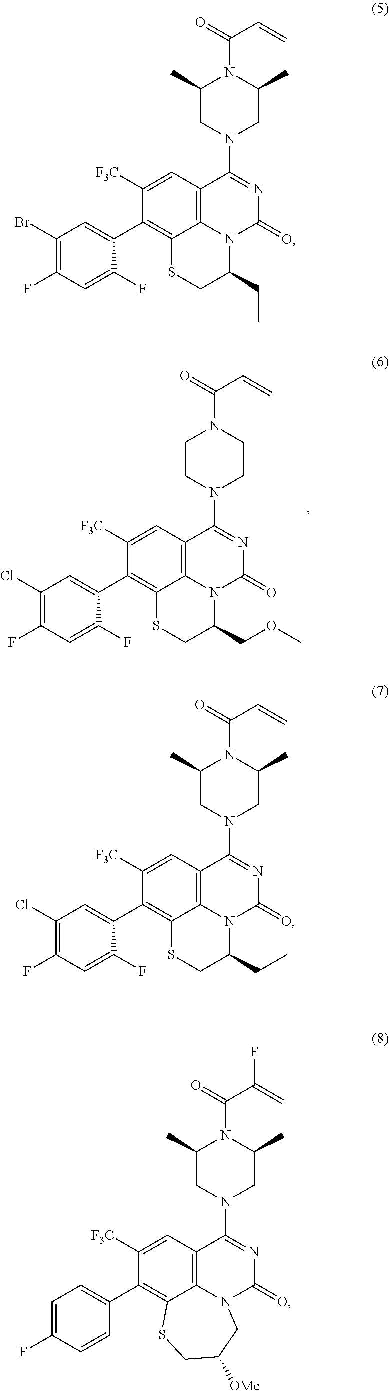

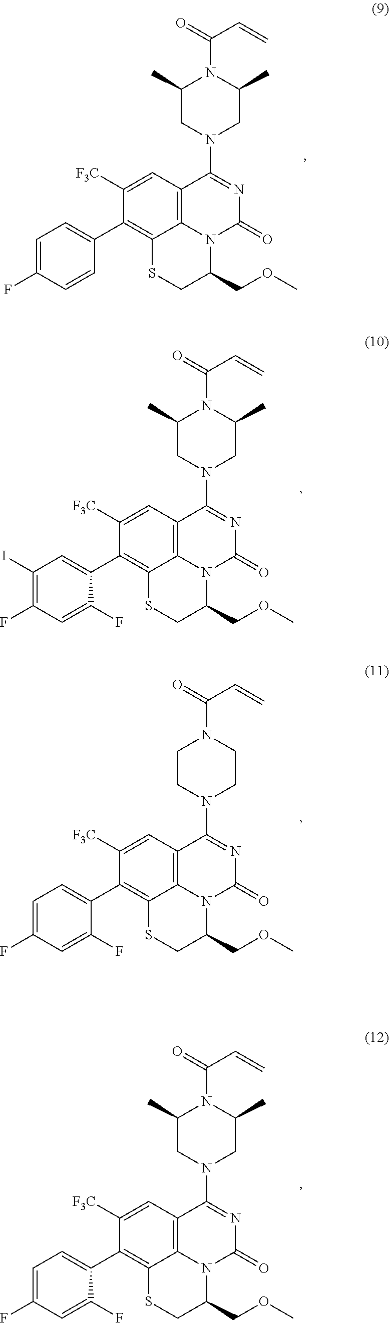

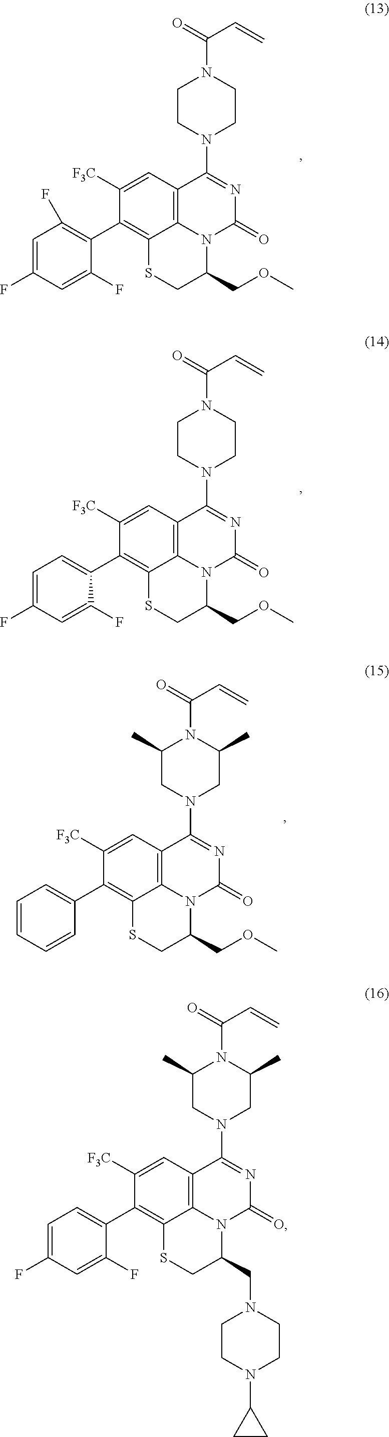

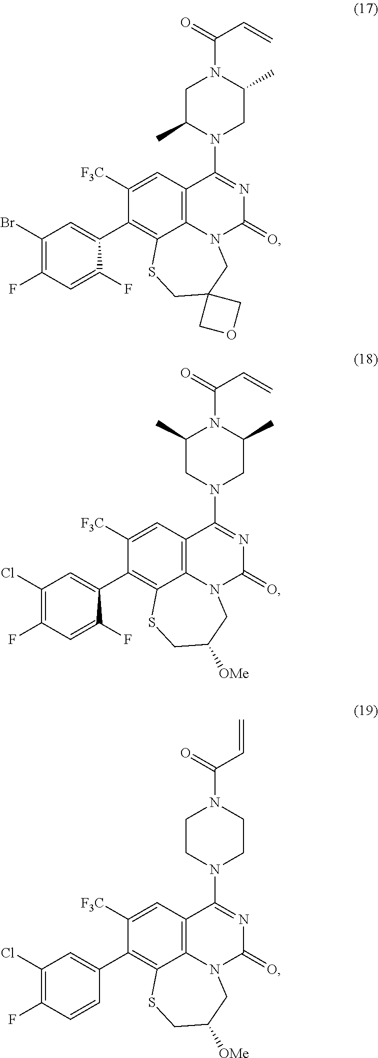

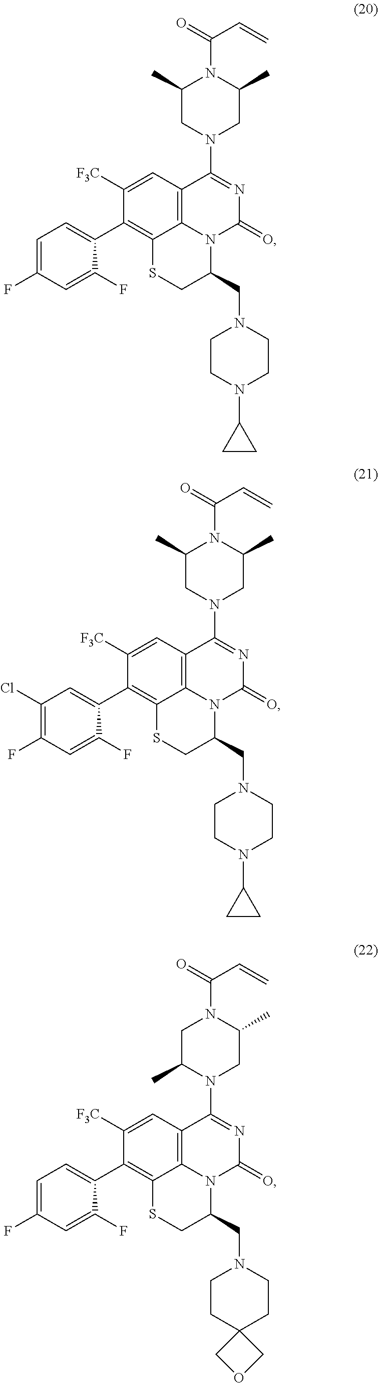

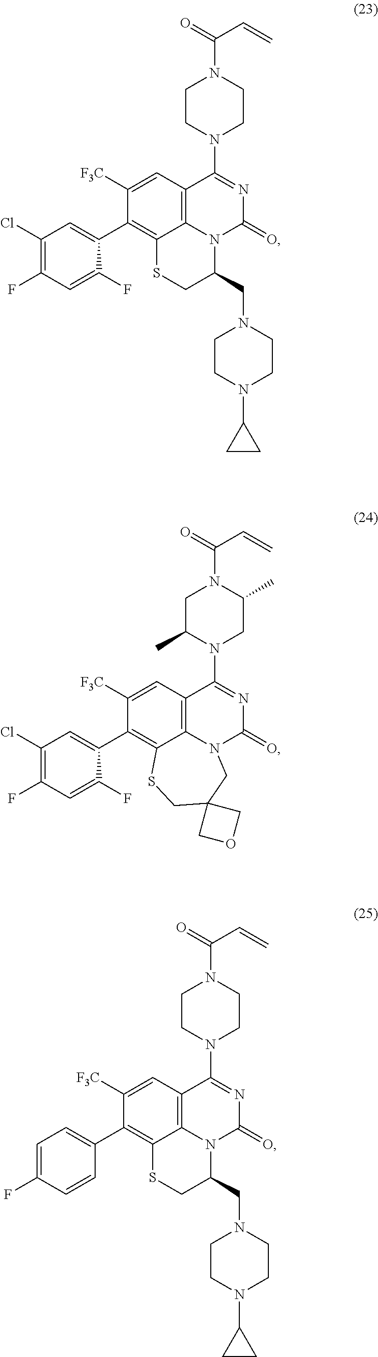

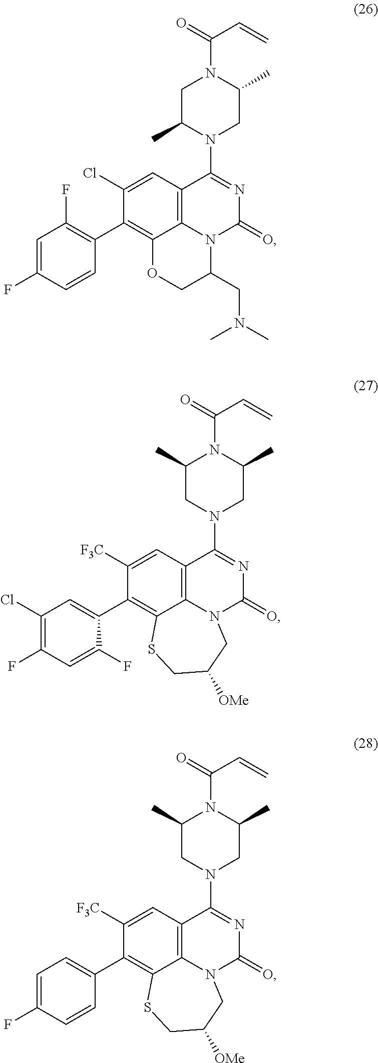

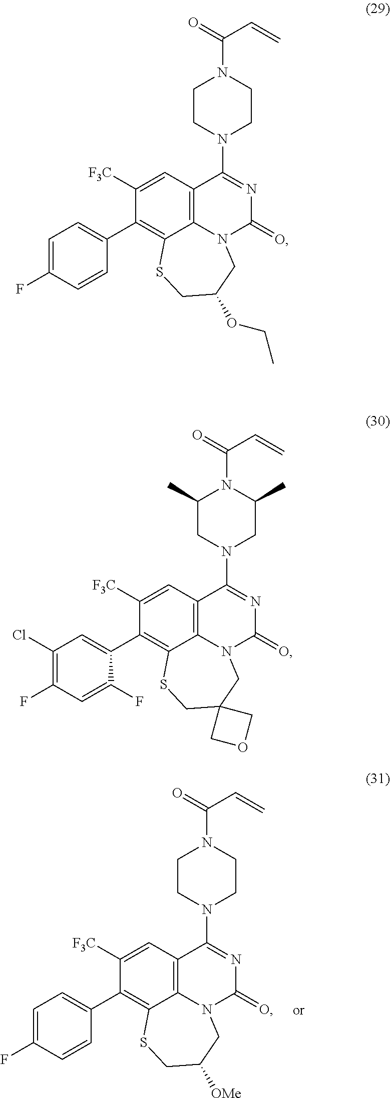

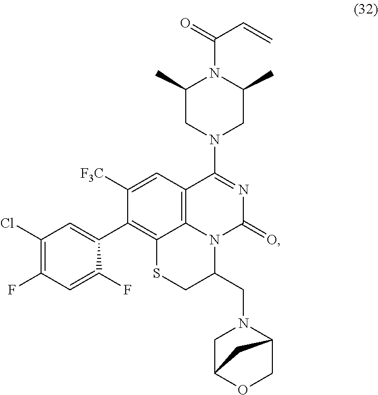

or a pharmaceutically acceptable salt or solvate thereof.

In some embodiments, other suitable KRAS G12C inhibitors are selected from:

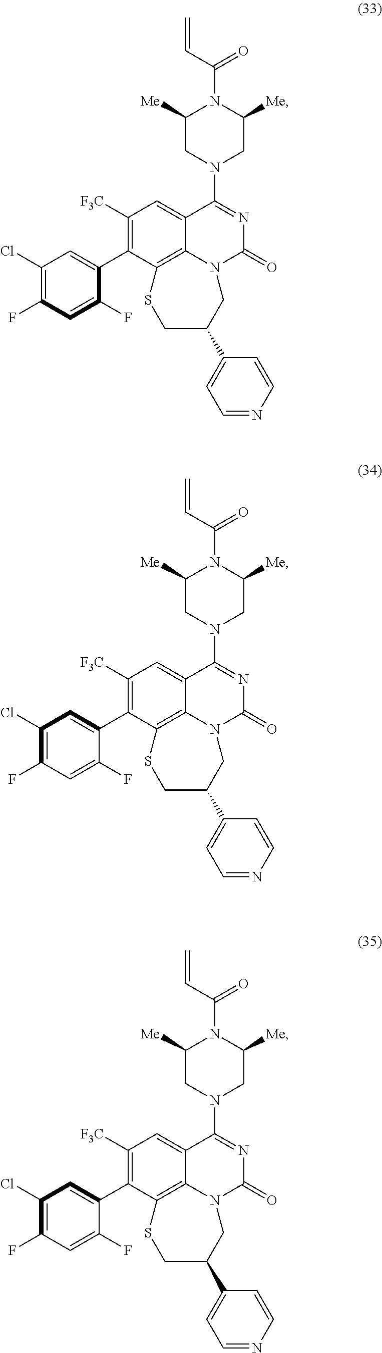

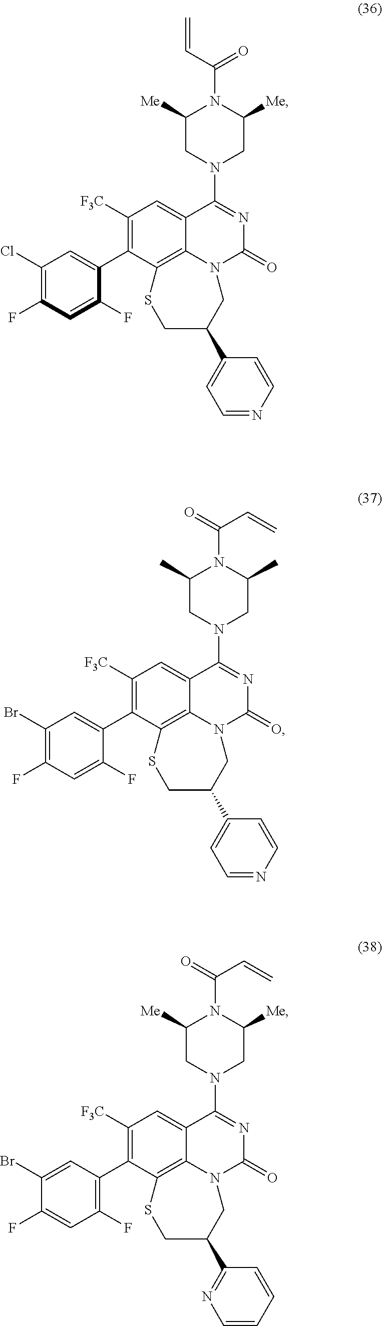

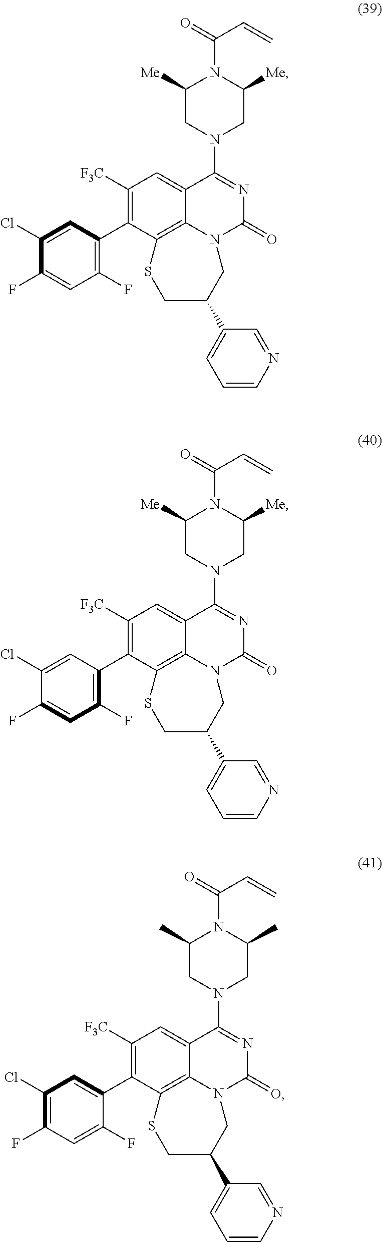

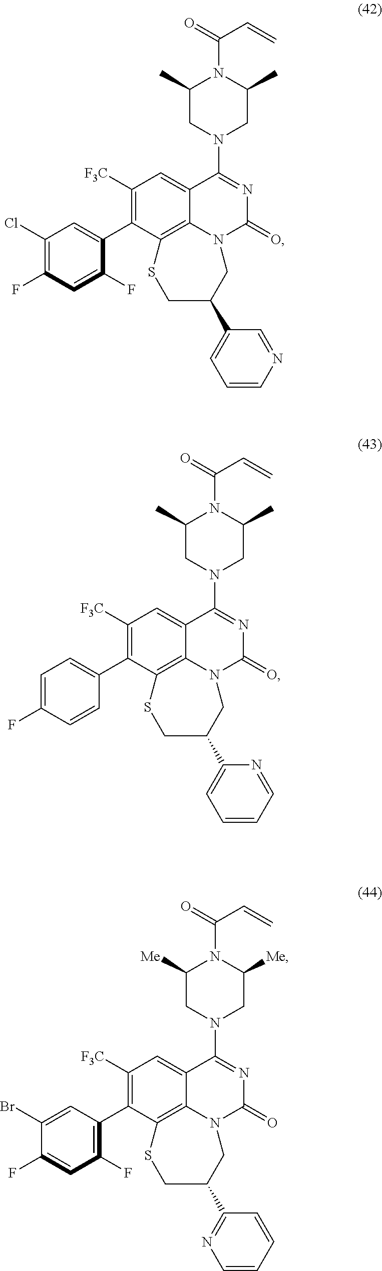

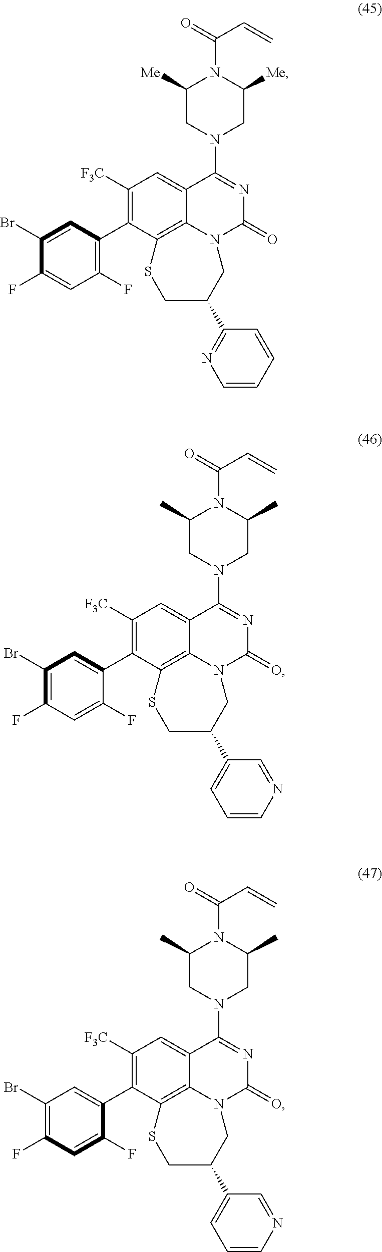

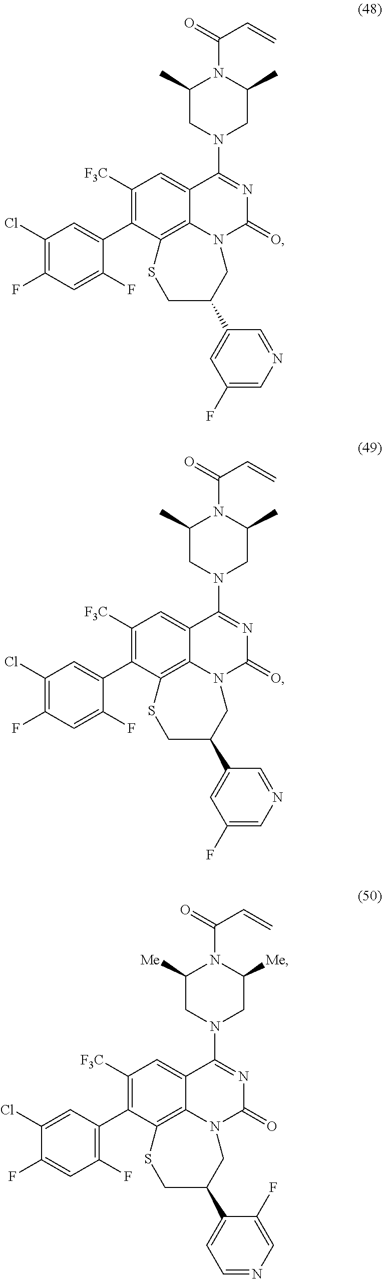 [SMILES: C=CC(=O)N1[C@H](C)CN(c2nc(=O)n3c4c(c(-c5cc(Cl)c(F)cc5F)c(C(F)(F)F)cc24)SC[C@@H](c2cncc(F)c2)C3)C[C@@H]1C ; C=CC(=O)N1[C@H](C)CN(c2nc(=O)n3c4c(c(-c5cc(Cl)c(F)cc5F)c(C(F)(F)F)cc24)SC[C@H](c2cncc(F)c2)C3)C[C@@H]1C ; C=CC(=O)N1[C@H](C)CN(c2nc(=O)n3c4c(c(-c5cc(Cl)c(F)cc5F)c(C(F)(F)F)cc24)SC[C@H](c2ccncc2F)C3)C[C@@H]1C]

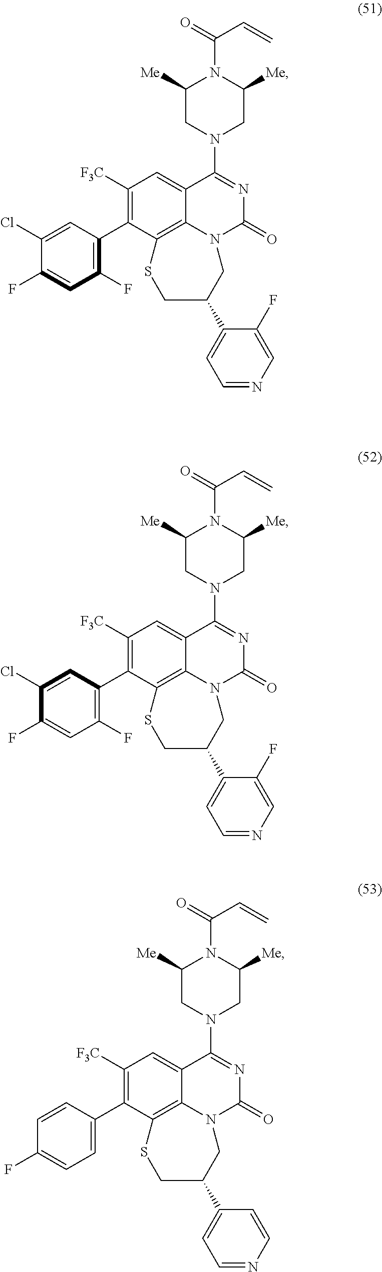

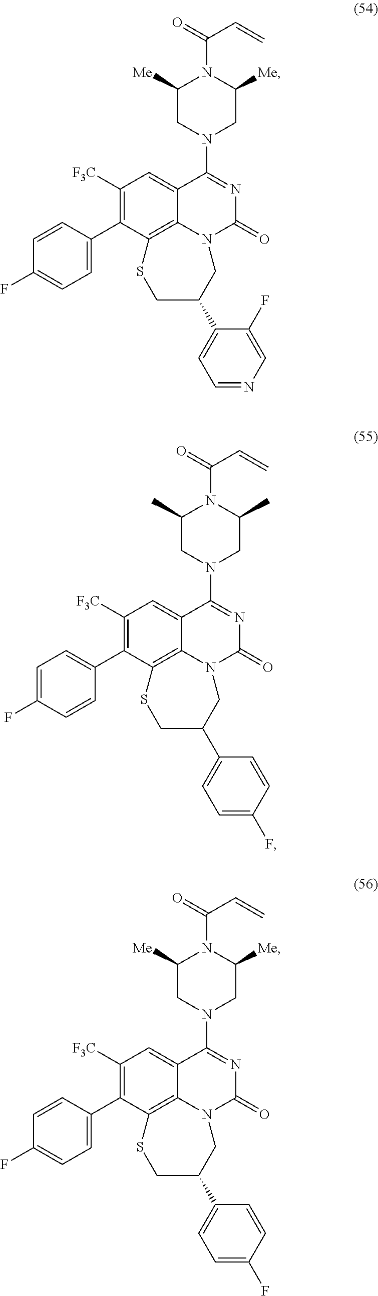 [SMILES: C=CC(=O)N1[C@H](C)CN(c2nc(=O)n3c4c(c(-c5ccc(F)cc5)c(C(F)(F)F)cc24)SC[C@@H](c2ccncc2F)C3)C[C@@H]1C ; C=CC(=O)N1[C@H](C)CN(c2nc(=O)n3c4c(c(-c5ccc(F)cc5)c(C(F)(F)F)cc24)SCC(c2ccc(F)cc2)C3)C[C@@H]1C ; C=CC(=O)N1[C@H](C)CN(c2nc(=O)n3c4c(c(-c5ccc(F)cc5)c(C(F)(F)F)cc24)SC[C@@H](c2ccc(F)cc2)C3)C[C@@H]1C]

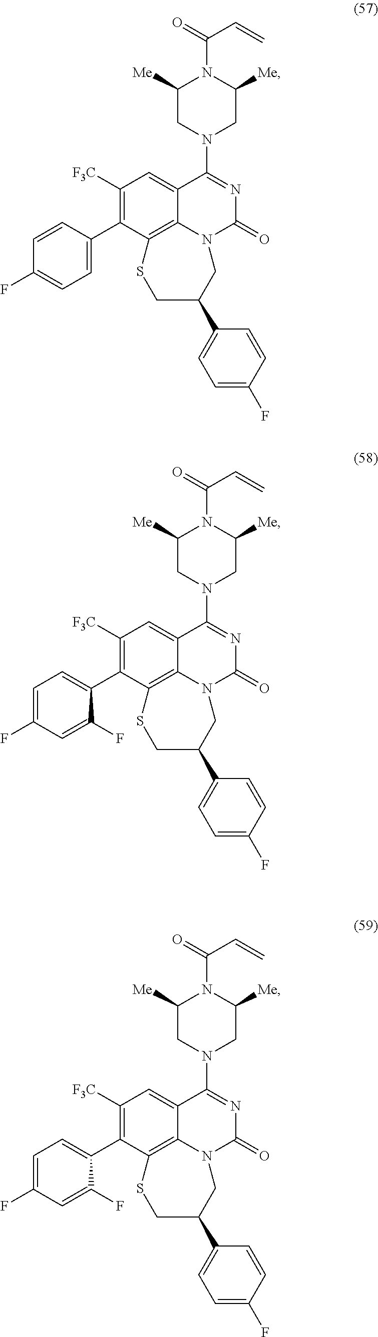

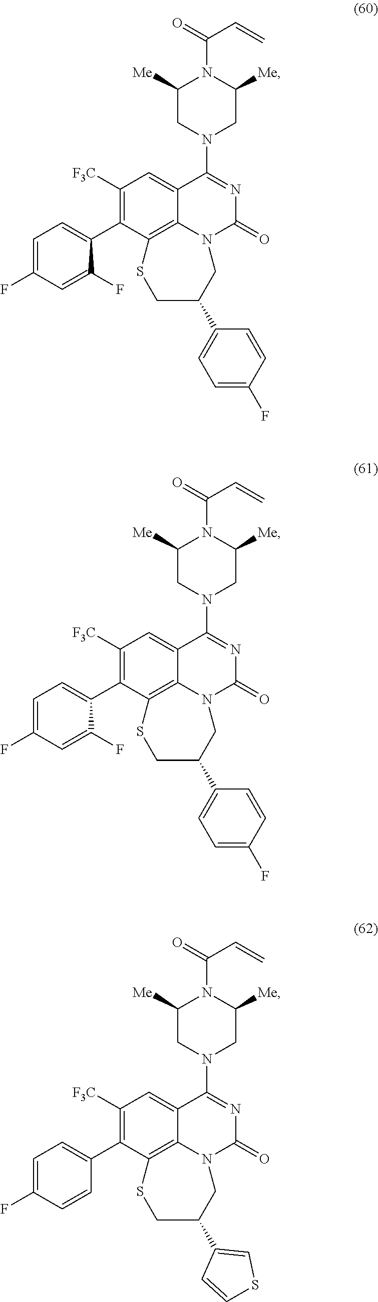

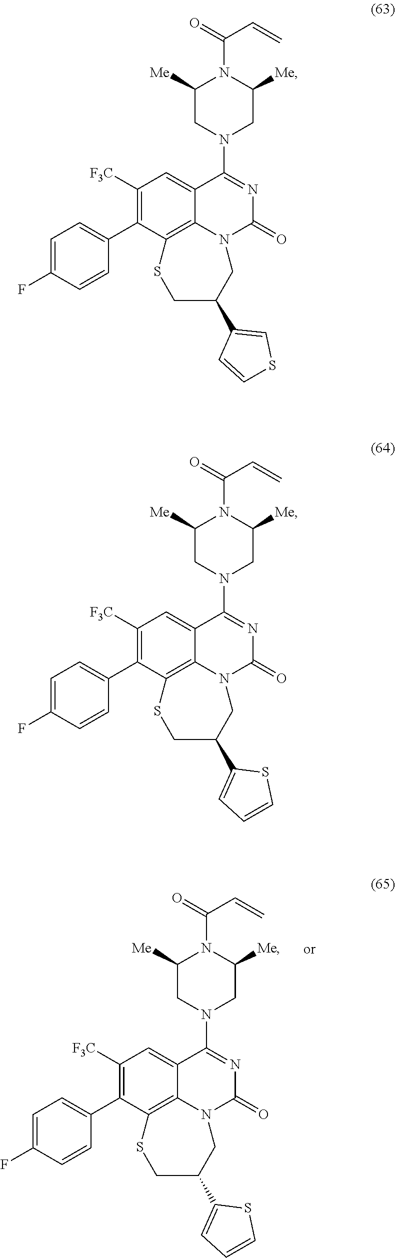 [SMILES: C=CC(=O)N1[C@H](C)CN(c2nc(=O)n3c4c(c(-c5ccc(F)cc5)c(C(F)(F)F)cc24)SC[C@H](c2ccsc2)C3)C[C@@H]1C ; C=CC(=O)N1[C@H](C)CN(c2nc(=O)n3c4c(c(-c5ccc(F)cc5)c(C(F)(F)F)cc24)SC[C@H](c2cccs2)C3)C[C@@H]1C ; C=CC(=O)N1[C@H](C)CN(c2nc(=O)n3c4c(c(-c5ccc(F)cc5)c(C(F)(F)F)cc24)SC[C@@H](c2cccs2)C3)C[C@@H]1C]

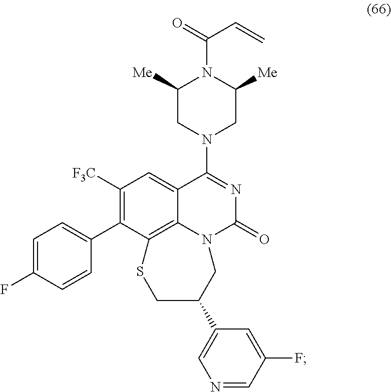 [SMILES: C=CC(=O)N1[C@H](C)CN(c2nc(=O)n3c4c(c(-c5ccc(F)cc5)c(C(F)(F)F)cc24)SC[C@@H](c2cncc(F)c2)C3)C[C@@H]1C]

or a pharmaceutically acceptable salt or solvate thereof.

### Trametinib

Trametinib:

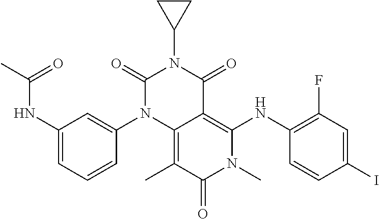 [SMILES: CC(=O)Nc1cccc(-n2c(=O)n(C3CC3)c(=O)c3c(Nc4ccc(I)cc4F)n(C)c(=O)c(C)c32)c1]

belongs to a class of pyrimidine compounds that are known mitogen-activated protein (MAP) kinase/extracellular signal-regulated (ERK) kinase (hereinafter referred to as MEK) inhibitors, for instance, MEK1 and MEK2. MEK inhibitory activity effectively induces inhibition of ERK1/2 and suppression of cell proliferation, which shows effects on diseases caused by undesirable cell proliferation, such as tumors and the like.

The terms “combination,” “therapeutic combination” or “pharmaceutical combination” as used herein refer to either a fixed combination in one dosage unit form, or non-fixed combination, or a kit of parts for the combined administration where two or more therapeutic agents may be administered together, independently at the same time or separately within time intervals, especially where these time intervals allow that the combination partners show a cooperative, e.g., synergistic, effect.

The term “combination therapy” refers to the administration of two or more therapeutic agents to treat a therapeutic condition or disorder described in the present disclosure. Such administration encompasses co-administration of these therapeutic agents in a substantially simultaneous manner, such as in a single formulation having a fixed ratio of active ingredients or in separate formulations (e.g., capsules and/or intravenous formulations) for each active ingredient. In addition, such administration also encompasses use of each type of therapeutic agent in a sequential or separate manner, either at approximately the same time or at different times. Regardless of whether the active ingredients are administered as a single formulation or in separate formulations, the drugs are administered to the same patient as part of the same course of therapy. In any case, the treatment regimen will provide beneficial effects in treating the conditions or disorders described herein.

In some embodiments the combination is for simultaneous, sequential, or separate administration. By simultaneous therapeutic use, within the meaning of the present embodiments is meant an administration of at least two active ingredients by the same route and at the same time or at substantially the same time.

By separate use, within the meaning of the present embodiments is meant in particular an administration of at least two active ingredients at the same time or at substantially the same time by different routes.

By sequential therapeutic use is meant administration of at least two active ingredients at different times, the administration route being identical or different. More particularly by an administration method is meant according to which the whole administration of one of the active ingredients is carried out before administration of the other or others commences.

In some embodiments, the combination is a fixed combination. In some embodiments, the combination is a non-fixed combination. The terms “fixed combination,” “fixed dose” and “single formulation” as used herein refers to a single carrier or vehicle or dosage form formulated to deliver an amount, which is jointly therapeutically effective for the treatment of cancer, of both therapeutic agents to a patient. The single vehicle is designed to deliver an amount of each of the agents along with any pharmaceutically acceptable carriers or excipients. In some embodiments, the vehicle is a tablet, capsule, pill, or a patch. In other embodiments, the vehicle is a solution or a suspension.

The term “non-fixed combination” or “kit of parts” means that the therapeutic agents of the combinations disclosed herein are both administered to a patient as separate entities either simultaneously, concurrently, or sequentially with no specific time limits, wherein such administration provides therapeutically effective levels of the two compounds in the body of a subject in need thereof. The latter also applies to cocktail therapy, e.g., the administration of three or more active ingredients.

The term “pharmaceutically acceptable” as used herein refers to those compounds, materials, compositions and/or dosage forms, which are, within the scope of sound medical judgment, suitable for contact with the tissues of a subject, e.g., a mammal or human, without excessive toxicity, irritation, allergic response and other problems or complications commensurate with a reasonable benefit/risk ratio.

As used herein, the term “pharmaceutically acceptable excipient” or “pharmaceutically acceptable carrier” includes any and all solvents, dispersion media, coatings, surfactants, antioxidants, preservatives (e.g., antibacterial agents, antifungal agents), isotonic agents, absorption delaying agents, salts, preservatives, drugs, drug stabilizers, binders, excipients, disintegration agents, lubricants, sweetening agents, flavoring agents, dyes, and the like and combinations thereof, as would be known to those skilled in the art. Except insofar as any conventional carrier is incompatible with the active ingredient, its use in the therapeutic or pharmaceutical compositions is contemplated.

The term “pharmaceutical composition” is defined herein to refer to a mixture or solution containing at least one therapeutic agent to be administered to a subject, e.g., a mammal or human, in order or treat a particular disease or condition affecting the subject. The present pharmaceutical combinations can be formulated in suitable pharmaceutical compositions for enteral or parenteral administration, such as sugar-coated tablets, tablets, capsules or suppositories, or ampoules. If not indicated otherwise, these are prepared in a manner known per se, for example by means of various conventional mixing, comminution, direct compression, granulating, sugar-coating, dissolving, lyophilizing processes, or fabrication techniques readily apparent to those skilled in the art. It will be appreciated that the unit content of a combination partner contained in an individual dose of each dosage form need not in itself constitute an effective amount since the necessary effective amount may be reached by administration of a plurality of dosage units. The pharmaceutical composition may contain, from about 0.1% to about 99.9%, or from about 1% to about 60%, of the therapeutic agent(s). One of ordinary skill in the art may select one or more of the aforementioned carriers with respect to the particular desired properties of the dosage form by routine experimentation and without any undue burden. The amount of each carriers used may vary within ranges conventional in the art. The following references disclose techniques and excipients used to formulate oral dosage forms: The Handbook of Pharmaceutical Excipients, 4th edition, Rowe et al., Eds., American Pharmaceuticals Association (2003); and Remington: the Science and Practice of Pharmacy, 20th edition, Gennaro, Ed., Lippincott Williams & Wilkins (2003). These optional additional conventional carriers may be incorporated into the oral dosage form either by incorporating the one or more conventional carriers into the initial mixture before or during granulation or by combining one or more conventional carriers with granules comprising the combination of agents or individual agents of the combination of agents in the oral dosage form. In the latter embodiment, the combined mixture may be further blended, e.g., through a V-blender, and subsequently compressed or molded into a tablet, for example a monolithic tablet, encapsulated by a capsule, or filled into a sachet.

Pharmaceutical compositions may be presented in unit dose forms containing a predetermined amount of active ingredient per unit dose. In some embodiments, the unit dose includes one or more vehicles such that each vehicle includes an effective amount of at least one of the therapeutic agents along with pharmaceutically acceptable carriers and excipients. In some embodiments, the unit dose is one or more tablets, capsules, pills, injections, infusions, patches, or the like, administered to the patient at the same time. As is known to those skilled in the art, the amount of active ingredient per dose will depend on the condition being treated, the route of administration and the age, weight, and condition of the patient. In some embodiments, unit dosage compositions are those containing a daily dose or sub-dose, or an appropriate fraction thereof, of an active ingredient. Furthermore, such pharmaceutical compositions may be prepared by any of the methods well known in the pharmacy art.

The pharmaceutical compositions may include a “therapeutically effective amount” or “effective amount” of a compound disclosed herein. The term “pharmaceutically effective amount,” “therapeutically effective amount” or “clinically effective amount” of a combination of therapeutic agents is an amount sufficient, at dosages and for periods of time necessary, to provide an observable or clinically significant improvement over the baseline of clinically observable signs and symptoms of the disorders treated with the combination. A therapeutically effective amount may vary according to factors such as the disease state, age, sex, and weight of the individual. A therapeutically effective amount is also one in which any toxic or detrimental effects of the therapeutic agents are outweighed by therapeutically beneficial effects.

A “therapeutically effective dosage” can modulate a measurable parameter, such as tumor growth rate or disease progression in a desired manner. The ability of a compound to modulate a measurable parameter can be evaluated in an animal model system predictive of efficacy in human tumors to help establish suitable dosing levels and schedules. Alternatively, this property of a composition can be evaluated by examining the ability of the compound to modulate an undesired parameter by using in vitro assays known to the skilled practitioner.

The term “jointly therapeutically active” or “joint therapeutic effect” as used herein means that the therapeutic agents can be given jointly, separately, or sequentially in such time intervals that they prefer such that the subject, especially human, to be treated, still show an (which can be synergistic) interaction (joint therapeutic effect). Whether this is the case can, inter alia, be determined by following the blood levels of the compounds, showing that both compounds are present in the blood of the human to be treated at least during certain time intervals.

As used herein the term “agent” is understood to mean a substance that produces a desired effect in a tissue, system, animal, mammal, human, or other subject. It is also to be understood that an “agent” may be a single compound or a combination or composition of two or more compounds.

The term “proliferative disease” includes a cancer.

As used herein, the term “cancer” refers to a disease characterized by the undesired and uncontrolled growth of aberrant cells. Cancer cells can spread locally or through the bloodstream and lymphatic system to other parts of the body. As used herein, the term “cancer” or “tumor” includes premalignant, as well as malignant cancers and tumors. The term “cancer” is used herein to mean a broad spectrum of tumors, including all solid and hematological malignancies.

An “oral dosage form” includes a unit dosage form prescribed or intended for oral administration. As used herein, the terms “treat,” “treatment” and “treating” refer to the reduction or amelioration of the progression, severity and/or duration of a disorder, e.g., a proliferative disorder, or the amelioration of one or more symptoms, suitably of one or more discernible symptoms, of the disorder resulting from the administration of one or more therapies. In specific embodiments, the terms “treat,” “treatment” and “treating” refer to the amelioration of at least one measurable physical parameter of a proliferative disorder, such as growth of a tumor, not necessarily discernible by the patient. In other embodiments the terms “treat,” “treatment” and “treating” refer to the inhibition of the progression of a proliferative disorder, either physically by, e.g., stabilization of a discernible symptom, physiologically by, e.g., stabilization of a physical parameter, or both. In other embodiments the terms “treat,” “treatment” and “treating” refer to the reduction or stabilization of tumor size or cancerous cell count.

Within the meaning of the present disclosure, the term “treat” also denotes to arrest, delay the onset (i.e., the period prior to clinical manifestation of a disease) and/or reduce the risk of developing or worsening a disease. The term “protect” is used herein to mean prevent, delay, or treat, or all, as appropriate, development, continuance, or aggravation of a disease in a subject, e.g., a mammal or human.

The term “subject” or “patient” as used herein is intended to include animals, which are capable of suffering from or afflicted with a cancer or any disorder involving, directly or indirectly, a cancer. Examples of subjects include mammals, e.g., humans, apes, monkeys, dogs, cows, horses, pigs, sheep, goats, cats, mice, rabbits, rats, and transgenic non-human animals. In some embodiments, the subject is a human, e.g., a human suffering from, at risk of suffering from, or potentially capable of suffering from a proliferative disease, such as cancer.

The term “inhibition,” “inhibitor,” or “antagonist” includes a reduction in a certain parameter, e.g., an activity, of a given molecule or pathway. For example, inhibition of an activity of a targeted kinase (Raf or KRAS G12C) by 5%, 10%, 20%, 30%, 40% or more is included by this term. Thus, inhibition may be, but need not be, 100%.

As used herein, “salts” (which, what is meant by “or salts thereof or “or a salt thereof), can be present alone or in mixture with free compounds of the combinations disclosed herein, e.g., a Raf inhibitor which is a Compound with formula (I), KRAS G12C inhibitor, and/or trametinib, and including pharmaceutically acceptable salts. Such salts are formed, for example, as acid addition salts, with organic or inorganic acids, from compounds of the combinations disclosed herein with a basic nitrogen atom, especially the pharmaceutically acceptable salts. The term “pharmaceutically acceptable salts” refers to salts that retain the biological effectiveness and properties of the compound and which typically are not biologically or otherwise undesirable. The compound may be capable of forming acid addition salts by virtue of the presence of an amino group.

Lists of suitable salts can be found, e.g., in “Remington's Pharmaceutical Sciences,” 20th ed., Mack Publishing Company, Easton, Pa., (1985); and in “Handbook of Pharmaceutical Salts: Properties, Selection, and Use” by Stahl and Wermuth (Wiley-VCH, Weinheim, Germany, 2002).

For isolation or purification purposes it is also possible to use pharmaceutically unacceptable salts, for example picrates or perchlorates. For therapeutic use, only pharmaceutically acceptable salts or free compounds can be employed (where applicable in the form of pharmaceutical preparations). In view of the close relationship between the novel compounds in free form and those in the form of their salts, including those salts that can be used as intermediates, for example in the purification or identification of the novel compounds, any reference to the free compounds is to be understood as referring also to the corresponding salts, as appropriate and expedient. The salts of compounds used in the combinations disclosed herein can be pharmaceutically acceptable salts; suitable counter-ions forming pharmaceutically acceptable salts are known in the field. Unless otherwise specified, or clearly indicated by the text, reference to therapeutic agents useful in the pharmaceutical combination provided herein includes both the free base of the compounds, and all pharmaceutically acceptable salts of the compounds.

As used herein, the term “solvate” refers to a complex of variable stoichiometry formed by a solute or a salt thereof and a solvent. Such solvents for the purpose of embodiments herein may not interfere with the biological activity of the solute. Examples of suitable solvents include, but are not limited to, water, methanol, dimethylsulfoxide, ethanol, and acetic acid. Examples of suitable pharmaceutically acceptable solvents include, without limitation, water, ethanol, and acetic acid.

The term “synergistic effect” as used herein, refers to action of two agents such as, for example, a Raf inhibitor which is a Compound with formula (I), or a pharmaceutically acceptable salt thereof, and a KRAS G12C inhibitor, or a pharmaceutically acceptable salt thereof, to produce an effect, for example, slowing the symptomatic progression of cancer or symptoms thereof, which is greater than the simple addition of the effects of each drug administered by themselves.

In some embodiments, there are provided pharmaceutical compositions comprising the pharmaceutical combination disclosed herein and at least one pharmaceutically acceptable carrier, as described herein above.

In some embodiments, there are provided pharmaceutical combinations or the pharmaceutical composition, as disclosed herein, for use in the treatment of a cancer.

In some embodiments, the cancer expresses a MAPK mutation or wherein the cancer is N-RAS mutant, H-RAS mutant or K-RAS mutant, or combination thereof. Included are KRAS-mutant cancers or tumors. The term “KRAS-mutant” tumor or cancer includes any tumor that exhibits a mutated KRAS protein, in particular gain of function KRAS-mutation; especially any G12X, G13X, Q61X or A146X KRAS-mutant, where X is any amino acid other than the one naturally occurring at that position. E.g., a G12V mutation means that a Glycine is substituted with Valine at codon 12. Examples of KRAS mutations in tumors include Q61H, Q61K, G12V, G12C, G12D, G12R, G12S, G13D, and A146T. Thus KRAS-mutant NSCLC includes tumors having at least one KRAS mutation corresponding to G12X, G13X, Q61X or A146X, particularly at least one KRAS mutation selected from Q61K, G12V, G12C and A146T NSCLC. The cancer may be at an early, intermediate, or late stage. KRAS-mutant cancers include KRAS G12D-mutant ovarian cancer; KRAS G12V-mutant or G13D-mutant colorectal cancer; KRAS Q61H-mutant, KRAS Q61K-mutant, KRAS G12C-mutant, KRAS G12S-mutant or KRAS G12V-mutant NSCLC; KRAS G12D-mutant, G12V-mutant, or KRAS G12R-mutant pancreatic cancer. Included are NRAS mutant cancers or tumors. The term “NRAS-mutant” tumor or cancer includes any tumor that exhibits a mutated NRAS protein, in particular gain of function NRAS-mutation; especially any G13R, Q61K, Q61L, Q61R, NRAS-mutant tumor. Thus NRAS-mutant melanoma includes melanoma having at least one NRAS mutation corresponding to Q61K, Q61L or Q61R. The cancer may be NRAS QG13R-mutant melanoma. The cancer may be at an early, intermediate, or late stage. The cancer may be locally advanced or metastatic.

In some embodiments, the cancer comprises a mutation at Q61 selected from Q61R, Q61L, and Q61M.

In some embodiments, the cancer is non-small cell lung cancer (NSCLC), colorectal cancer (CRC), or pancreatic ductal adenocarcinoma (PDAC).

In some embodiments, the cancer is colorectal cancer (CRC).

In some embodiments, the cancer is pancreatic ductal adenocarcinoma (PDAC).

In some embodiments, the cancer is non-small cell lung cancer (NSCLC).

In some embodiments, the cancer is characterized by a mutation selected from the group consisting of BRAF, NRAS, KRAS mutation and combinations thereof.

In some embodiments, the cancer is selected from the group consisting of KRAS-mutant NSCLC (non-small cell lung cancer), KRAS-mutant colorectal cancer (CRC), and KRAS-mutant pancreatic cancer KRAS-mutant pancreatic ductal adenocarcinoma (PDAC).

In some embodiments, the pharmaceutical combination or the pharmaceutical composition further comprises an anti-PD-1, anti-PD-L1, or anti-EGFR antibody.

In some embodiments, there are provided methods of treating cancer expressing a MAPK mutation or wherein the cancer is N-RAS mutant, H-RAS mutant or K-RAS mutant, or combination thereof, the method comprising administering the pharmaceutical combinations or the pharmaceutical compositions described herein.

In some embodiments, the cancer is non-small cell lung cancer (NSCLC), colorectal cancer (CRC), or pancreatic ductal adenocarcinoma (PDAC).

In some embodiments, the cancer is non-small cell lung cancer (NSCLC).

In some embodiments, the cancer is colorectal cancer (CRC).

In some embodiments, the cancer is selected from the group consisting of KRAS-mutant NSCLC (non-small cell lung cancer), KRAS-mutant colorectal cancer (CRC), and KRAS-mutant pancreatic cancer KRAS-mutant pancreatic ductal adenocarcinoma (PDAC).

In some embodiments, the cancer comprises a mutation at Q61 selected from Q61R, Q61L, and Q61M.

In some embodiments, the cancer comprises NF-1 Loss of Function.

In some embodiments, the cancer comprises a RAS G13R mutation.

In some embodiments, the cancer comprises a KRAS G12C mutation. In some such embodiments, the cancer is non-small cell lung cancer (NSCLC).

In some embodiments, the cancer comprises a Class 2 BRAF mutation.

In some embodiments, the cancer comprises a Class 3 BRAF mutation.

In some embodiments, the pharmaceutical combination or the pharmaceutical composition further comprises an anti-PD-1, anti-PD-L1, or anti-EGFR antibody. For example, suitable pharmaceutical combinations of compositions may comprise an anti-PD-1 antibody including, without limitation, pembrolizumab, nivolumab, pidilizumab, cemiplimab, SHR-1210, PDR001, or AMP-224. Suitable PD-L1 antibodies may include, without limitation, atezolizumab, avelumab, durvalumab, BMS-935559, MED14736, MPDL3280A (also known as RG7446), or MSB0010718C. Suitable EGFR antibodies include, without limitation, cetuximab, panitumumab, nimotuzumab, or necitumumab.

By way of non-limiting example, a triple pharmaceutical combination or composition to treat KRAS G12C mutant lung cancer, including non-small cell lung cancer (NSCLC), may comprise the RAF inhibitor: compound of formula (I), plus sotorasib, and pembrolizumab.

By way of further non-limiting example, a triple pharmaceutical combination or composition to treat KRAS G12C mutant colon cancer, including colorectal cancer (CRC), may comprise the RAF inhibitor: compound of formula (I), plus sotorasib, and cetuximab.

By way of yet further non-limiting example, a triple pharmaceutical combination or composition to treat KRAS G12C mutant pancreatic cancer, including pancreatic ductal adenocarcinoma (PDAC), may comprise the RAF inhibitor: compound of formula (I), plus sotorasib, and panitumumab.

In some embodiments, the Raf inhibitor is Compound of formula (I), as defined herein, or a pharmaceutically acceptable salt thereof, is combined with a KRAS G12C inhibitor, or a pharmaceutically acceptable salt or solvate thereof, and may be administered at a therapeutic or lower-than therapeutic dose relative to a single-agent dose level. In some embodiments, the concentration or dosage of the one therapeutic agent that is required to achieve inhibition, e.g., growth inhibition or tumor shrinkage is lower when the other therapeutic agent is used or administered in combination with the first therapeutic agent than when each therapeutic agent is administered individually. In some embodiments, in a combination therapy, the concentration or dosage of one therapeutic agent that is required to achieve inhibition, e.g., growth inhibition, is lower than the therapeutic dose as a monotherapy, e.g., 10-20%, 20-30%, 30-40%, 40-50%, 50-60%, 60-70%, 70-80%, or 80-90% lower.

In determining a synergistic interaction between one or more components, the optimum range for the effect and absolute dose ranges of each component for the effect may be definitively measured by administration of the components over different w/w ratio ranges and doses to patients in need of treatment. For humans, the complexity and cost of carrying out clinical studies on patients may render the use of this form of testing as a primary model for synergy impractical. However, the observation of synergy in certain experiments can be predictive of the effect in other species, and animal models exist that can be used to further quantify a synergistic effect. The observation of synergy in one species can be predictive of the effect in other species and using animal models, as described herein, a synergistic effect can be measured and the results of such studies can also be used to predict effective dose ratio ranges and the absolute doses and plasma concentrations required in other species by the application of pharmacokinetic/pharmacodynamic (PK/PD) methods. Established correlations between tumor models and effects seen in man suggest that synergy in animals may be demonstrated, for example, by xenograft models or in appropriate cell lines. It can be shown by established test models that combinations disclosed herein result in the beneficial effects described herein. The person skilled in the art is fully enabled to select a relevant test model to prove such beneficial effects. The pharmacological activity of the combinations disclosed herein may, for example, be demonstrated in a clinical study or in an in vivo or in vitro test procedure as essentially described herein.

Administration of the combination includes administration of the combination in a single formulation or unit dosage form, administration of the individual agents of the combination concurrently but separately, or administration of the individual agents of the combination sequentially by any suitable route. The individual combination partners of the combinations disclosed herein may be administered separately at different times during the course of therapy, or sequentially in any order or concurrently in divided or single combination forms, e.g., simultaneously or in jointly therapeutically effective amounts, including synergistically effective amounts, e.g., in daily or intermittent (i.e., not daily) dosages corresponding to the amounts described herein.

Compound of formula (I), or a pharmaceutically acceptable salt thereof, for use in the methods, treatments, combinations, and compositions disclosed herein are potent inhibitors of BRAF and CRAF. In some embodiments, Compound of formula (I), or a pharmaceutically acceptable salt thereof is administered orally. In one embodiment, Compound of formula (I), or a pharmaceutically acceptable salt thereof, is administered at a dose of about 50-1200 mg (e.g., per day). Compound of formula (I), or a pharmaceutically acceptable salt thereof, can be administered at a unit dosage of about 50 mg, about 100 mg, about 150 mg, about 200 mg, about 250 mg, about 300 mg, about 350 mg, about 400 mg, about 450 mg, about 500 mg about 550 mg, about 600 mg, about 650 mg, about 700 mg, about 750 mg, about 800 mg, about 850 mg, about 900 mg, about 950 mg, about 1000 mg, about 1050 mg, about 1100 mg, about 1150 mg or about 1200 mg. The unit dosage of Compound of formula (I), or a pharmaceutically acceptable salt thereof, may be administered once daily, or twice daily, or three times daily, or four times daily, with the actual dosage and timing of administration determined by criteria such as the patient's age, weight, and gender; the extent and severity of the cancer to be treated; and the judgment of a treating physician. In some embodiments, the unit dosage of Compound of formula (I) is administered once daily. In another embodiment, the unit dosage of Compound of formula (I) is administered twice daily.

In some embodiments, the KRAS G12C inhibitor, or a pharmaceutically acceptable salt thereof is administered orally. In one embodiment, the KRAS G12C inhibitor, or a pharmaceutically acceptable salt thereof, is administered at a dose of about 50-1200 mg (e.g., per day). The KRAS G12C inhibitor, or a pharmaceutically acceptable salt thereof, can be administered at a unit dosage of about 50 mg, about 100 mg, about 150 mg, about 200 mg, about 250 mg, about 300 mg, about 350 mg, about 400 mg, about 450 mg, about 500 mg about 550 mg, about 600 mg, about 650 mg, about 700 mg, about 750 mg, about 800 mg, about 850 mg, about 900 mg, about 950 mg, about 1000 mg, about 1050 mg, about 1100 mg, about 1150 mg or about 1200 mg. The unit dosage of KRAS G12C inhibitor, or a pharmaceutically acceptable salt thereof, may be administered once daily, or twice daily, or three times daily, or four times daily, with the actual dosage and timing of administration determined by criteria such as the patient's age, weight, and gender; the extent and severity of the cancer to be treated; and the judgment of a treating physician. In some embodiments, the unit dosage of KRAS G12C inhibitor is administered once daily. In another embodiment, the unit dosage of KRAS G12C inhibitor is administered twice daily.

In some embodiments, trametinib, or a pharmaceutically acceptable salt thereof is administered orally. In one embodiment, trametinib, or a pharmaceutically acceptable salt thereof, is administered at a dose of about 0.5-2.0 mg (e.g., per day). Trametinib, or a pharmaceutically acceptable salt thereof, can be administered at a unit dosage of about 0.5 mg, about 1.0 mg, or about 2.0 mg. The unit dosage of trametinib, or a pharmaceutically acceptable salt thereof, may be administered once daily, with the actual dosage and timing of administration determined by criteria such as the patient's age, weight, and gender; the extent and severity of the cancer to be treated; and the judgment of a treating physician. In some embodiments, the unit dosage of trametinib is administered once daily.

### EXAMPLES

### Example 1: Synergistic Combinations of Compound of Formula (I)

This Example demonstrates the synergistic combination of the compound of Formula I with KRAS G12C inhibitors.

Combination cellular proliferation assays: MIA PaCa-2 cells (5000 cells per well) were plated onto 96-well plates in 100 ul cell culture medium. Cells were treated with the compound of Formula I with either sotorasib or KRAS G12C inhibitor compound (12) at concentrations varying from 0 to 1 uM by using the Tecan D300e Digital Dispenser combination matrix protocol. At day 5, 100 ul of CellTiter-Glo (CTG) reagent. (Promega) was added and the plates were incubated for 60 minutes with gentle shaking. After 60 minutes of incubation, the luminescent signal was determined according to the provider's instructions (Promega) and combination data was generated by the standard HSA model using our proprietary combination analysis software. This was done in duplicate. The combination synergy was represented by positive numbers in results table. The negative numbers represent antagonism of the combination.

KRAS G12C inhibitor compound (12) has the following structure:

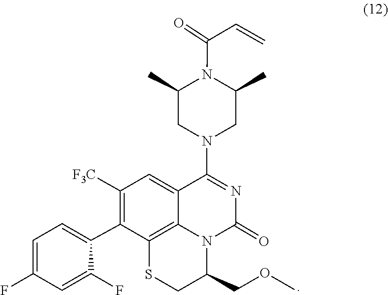

The results of these experiments are indicated in Tables 1 and 2 below.

TABLE 1
Naporafenib combination benefit with KRAS G12C inhibitor
Sotorasib in KRAS G12C mutant cell line, MIA PaCa−2
Sotorasib [nM]

| | | 0.05 | 0.15 | 0.46 | 1.37 | 4.12 | 12.35 | 37.04 | 111.1 | 333.3 | 1000 |
|---|---|---|---|---|---|---|---|---|---|---|---|
| Naporafenibb | 13.7 | −5.7 | −10.3 | −3.4 | −6.9 | −14.8 | −15.6 | −9.3 | −2.1 | −2.1 | −0.6 |
| [nM] | 41.2 | −4.5 | −12.1 | −5.4 | −3.7 | −12.4 | −12.0 | −2.0 | −0.1 | −1.0 | −0.6 |
| | 124 | −4.8 | −3.2 | −0.1 | 2.5 | −3.8 | −4.4 | 0.7 | −0.5 | −1.1 | −0.1 |
| | 370 | −0.1 | −2.1 | 1.2 | 5.2 | 11.4 | 10.7 | 5.7 | 1.9 | 0.1 | −0.7 |
| | 1111 | −5.8 | −4.9 | −3.2 | −2.0 | 4.9 | 17.3 | 10.7 | 4.1 | 1.1 | 1.8 |
| | 3333 | 2.0 | 2.9 | 3.7 | 4.0 | 4.7 | 9.8 | 15.7 | 10.7 | 6.2 | 5.6 |
| | 10000 | −1.0 | −0.9 | −0.9 | −0.5 | −0.3 | 0.2 | 1.1 | 2.0 | 2.4 | 3.0 |

N = 2; HSA synergy and antagonism; naporafenib vs sotorasib in Model MIA PaCa-2; ‘+’ synergy and ‘−’ antagonism

TABLE 2
Naporafenib combination benefit with KRAS G12C inhibitor
Compound (12) in KRAS G12C mutant cell line, MIA PaCa−2
Compound (12) [nM]

| | | 0.05 | 0.15 | 0.46 | 1.37 | 4.12 | 12.35 | 37.04 | 111.1 | 333.3 | 1000 |
|---|---|---|---|---|---|---|---|---|---|---|---|
| Naporafenib | 13.7 | −5.4 | −3.8 | −4.0 | −5.3 | −8.7 | −16.7 | −8.0 | −2.5 | −1.4 | −0.8 |
| [nM] | 41.2 | −2.0 | 1.2 | 0.2 | −1.6 | −2.5 | −7.3 | −1.8 | 0.3 | −0.4 | −0.7 |
| | 124 | −0.9 | −0.2 | 0.0 | 0.4 | 1.3 | 2.0 | 3.8 | 1.7 | −0.5 | −1.2 |
| | 370 | 3.4 | 5.0 | 5.8 | 11.3 | 12.8 | 14.3 | 8.9 | 3.3 | 0.0 | 0.0 |
| | 1111 | −0.4 | 1.2 | 4.4 | 2.6 | 8.5 | 20.8 | 16.7 | 5.9 | 1.8 | 1.7 |
| | 3333 | −0.5 | 2.1 | 0.1 | 3.1 | 4.1 | 7.3 | 10.2 | 14.4 | 8.2 | 7.2 |
| | 10000 | −2.0 | −1.4 | −1.3 | −0.6 | −0.7 | −0.2 | 0.9 | 2.5 | 3.8 | 4.5 |

N = 2; HSA synergy and antagonism; naporafenib vs compound (12) in Model MIA PaCa-2; ‘+’ synergy and ‘−’ antagonism

While specific embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A pharmaceutical combination comprising (a) a Raf inhibitor which is Compound of formula (I) 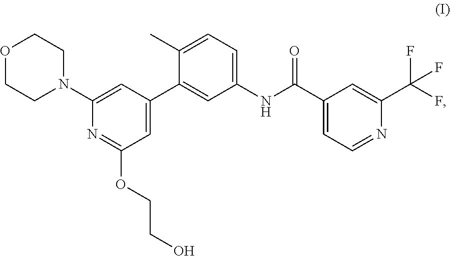 [SMILES: Cc1ccc(NC(=O)c2ccnc(C(F)(F)F)c2)cc1-c1cc(OCCO)nc(N2CCOCC2)c1]
or a pharmaceutically acceptable salt thereof,
and (b) a KRAS G12C inhibitor.

2. A pharmaceutical combination comprising (a) a Raf inhibitor which is Compound of formula (I) 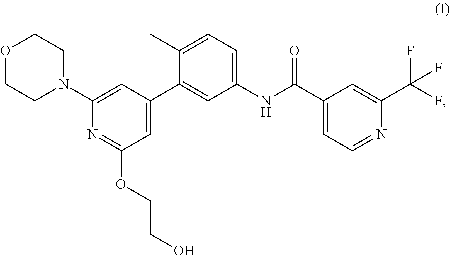 [SMILES: Cc1ccc(NC(=O)c2ccnc(C(F)(F)F)c2)cc1-c1cc(OCCO)nc(N2CCOCC2)c1]
or a pharmaceutically acceptable salt thereof,
(b) a KRAS G12C inhibitor,
and (c) trametinib.

3. The pharmaceutical combination according to claim 1 or 2, wherein the KRAS G12C inhibitor is selected from sotorasib, adagrasib, 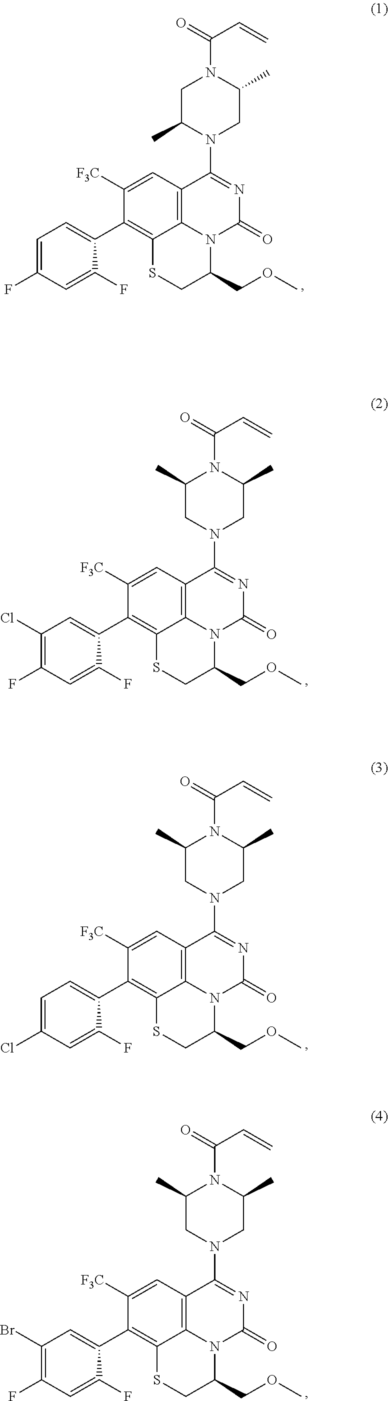 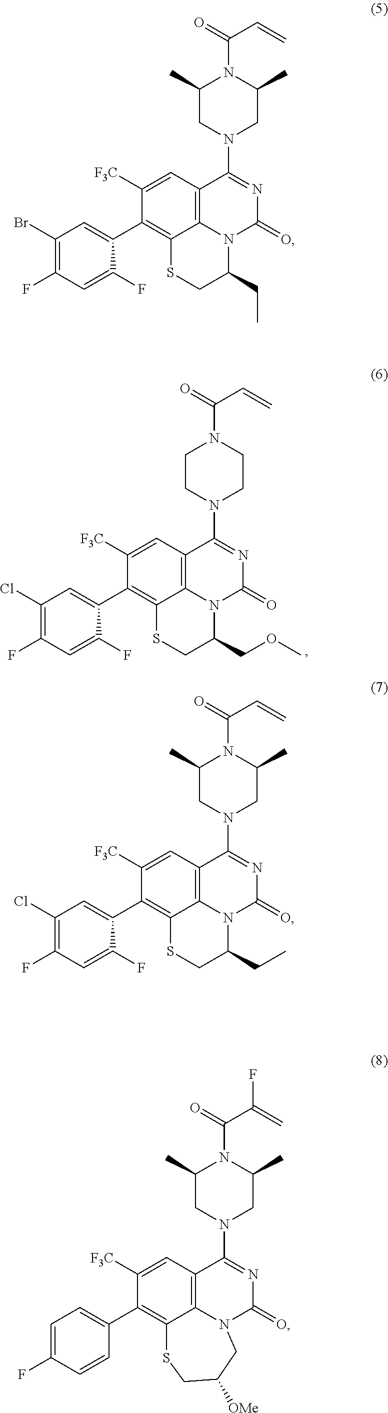 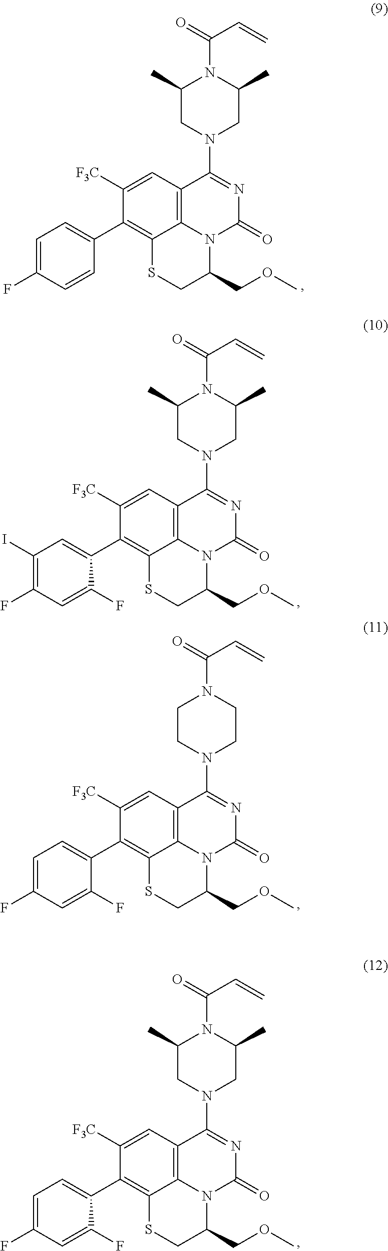 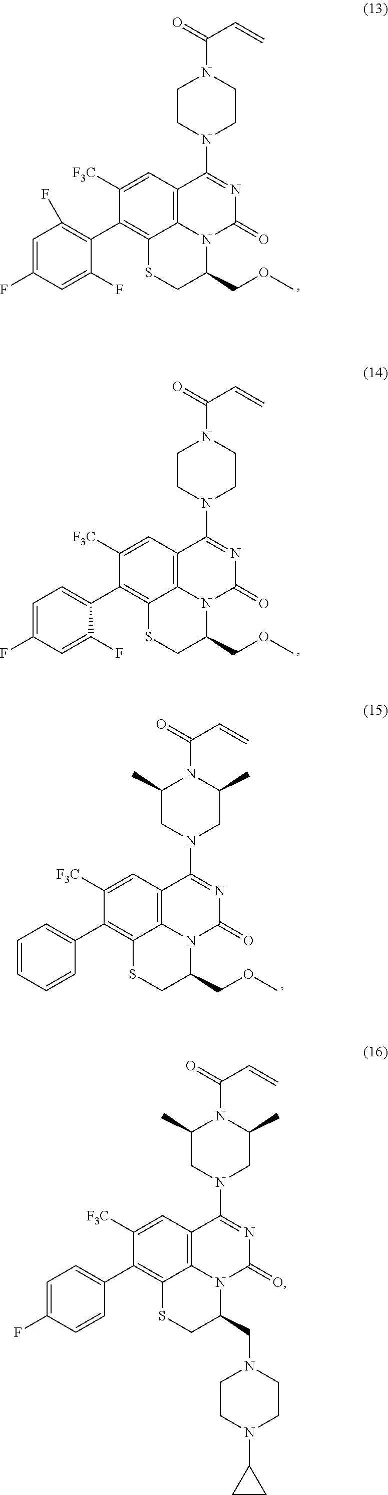 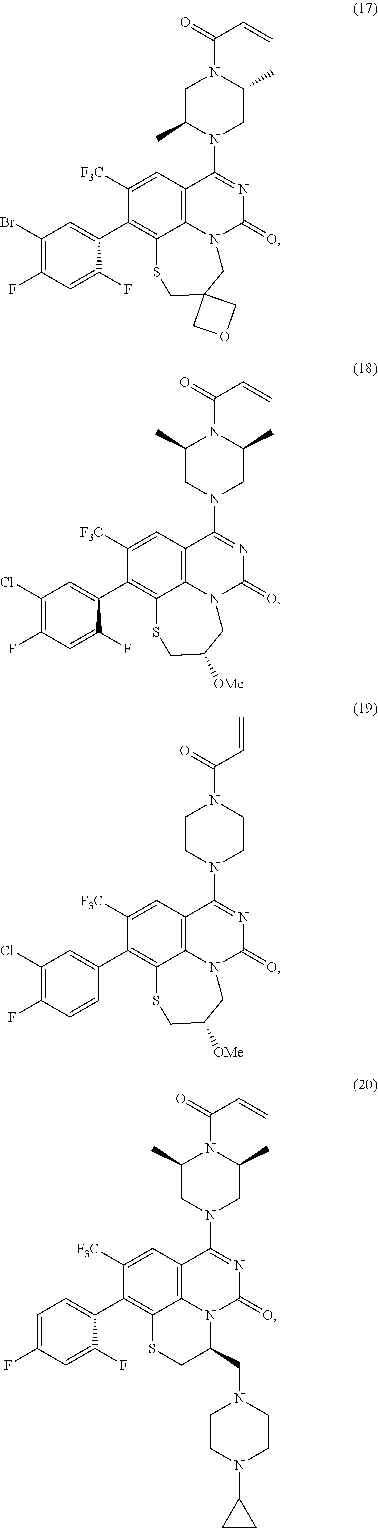 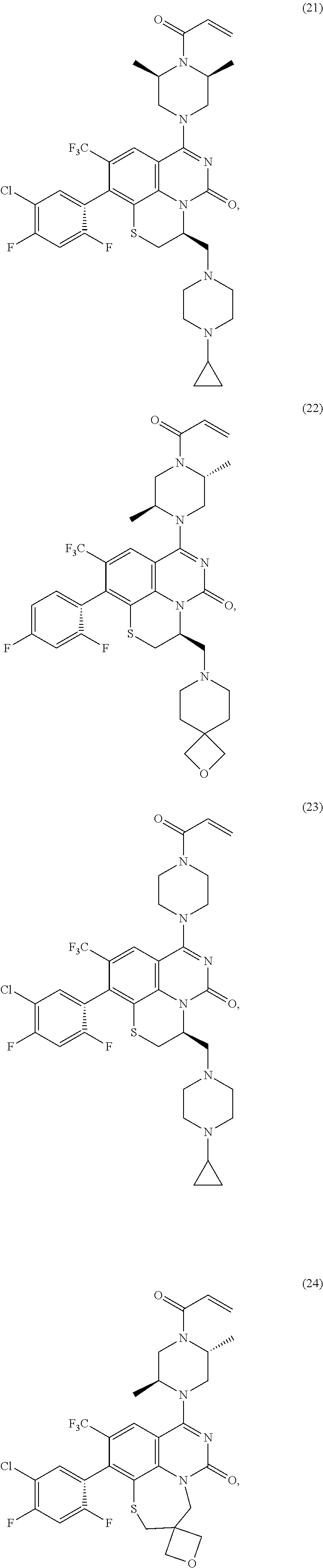 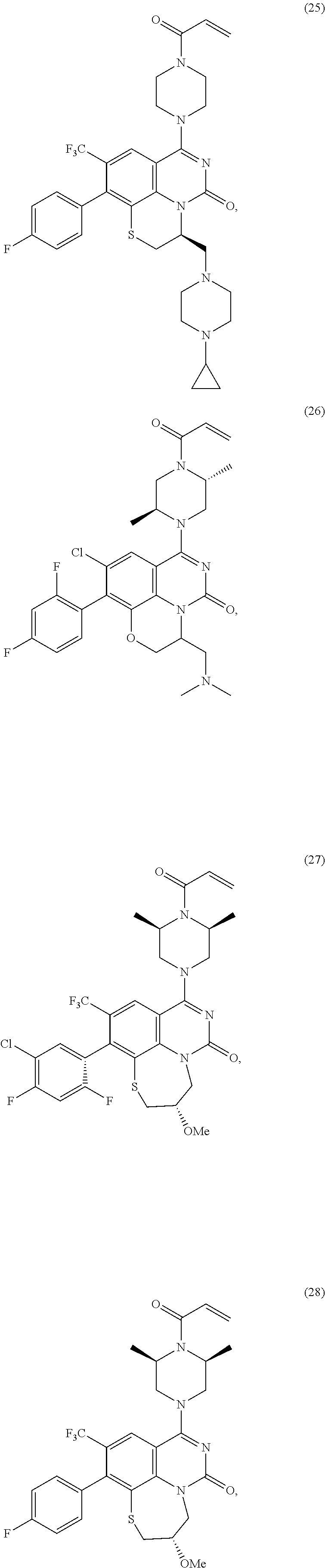 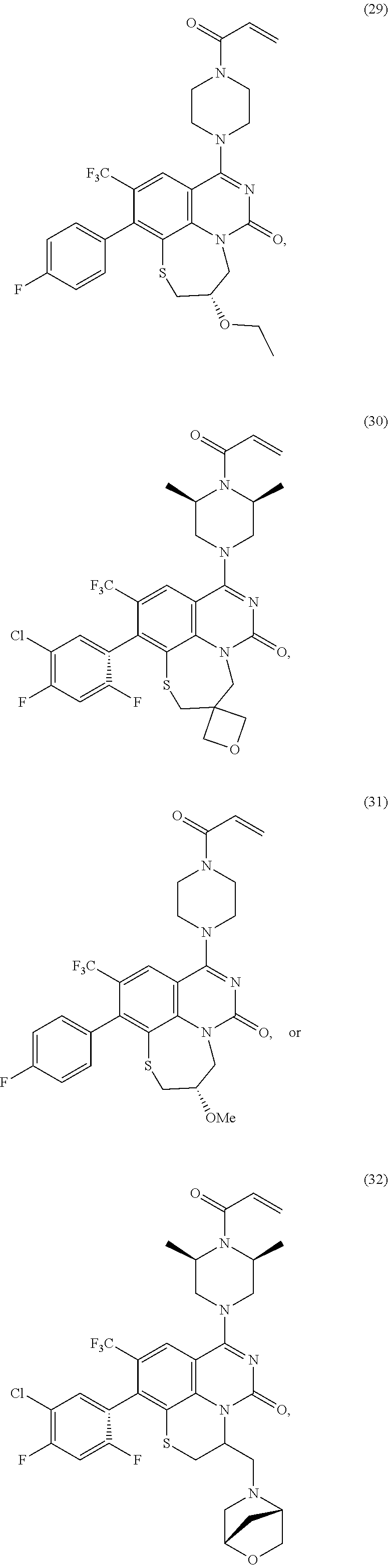
or a pharmaceutically acceptable salt or solvate thereof.

4. The pharmaceutical combination according to any one of claims 1 to 3, wherein the KRAS G12C inhibitor inhibitor is selected from: 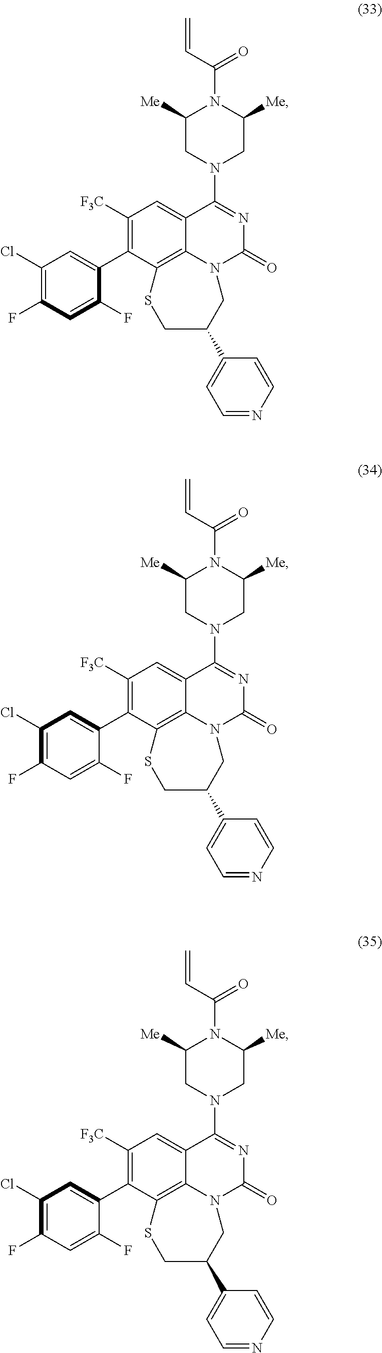 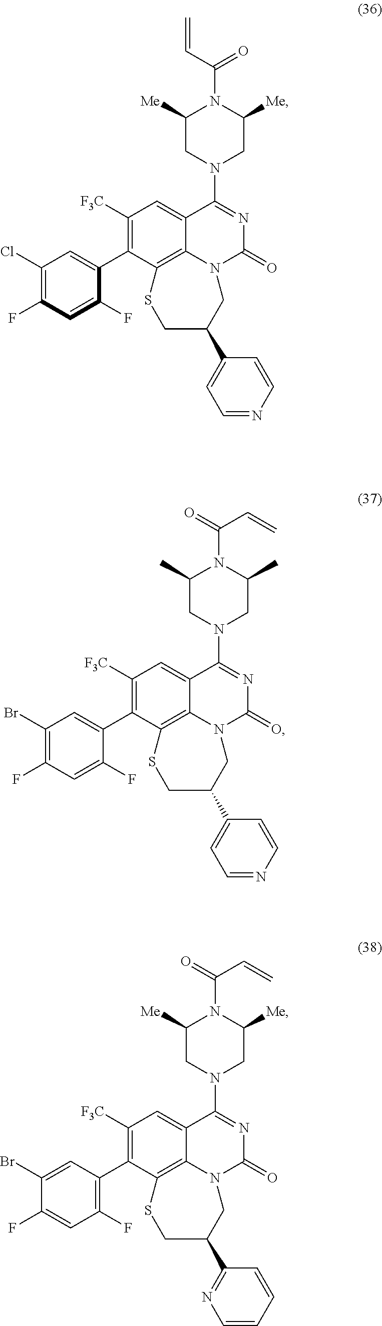 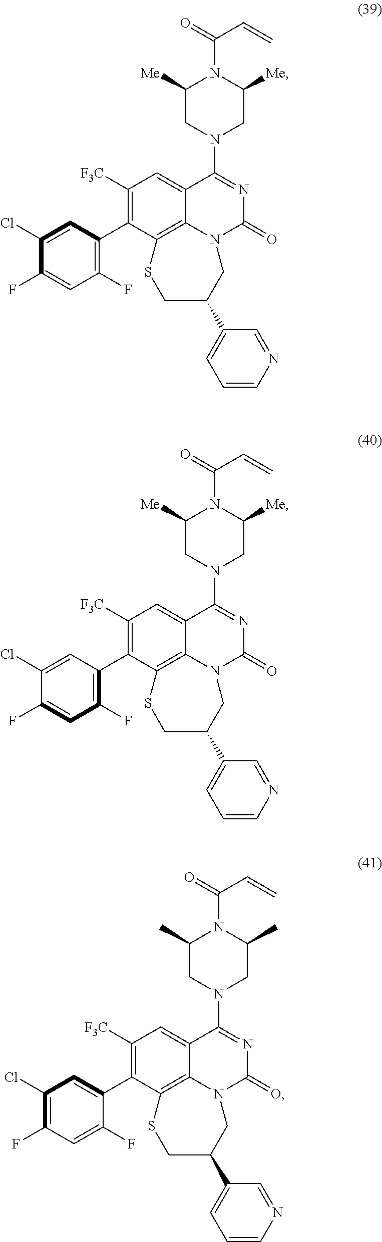 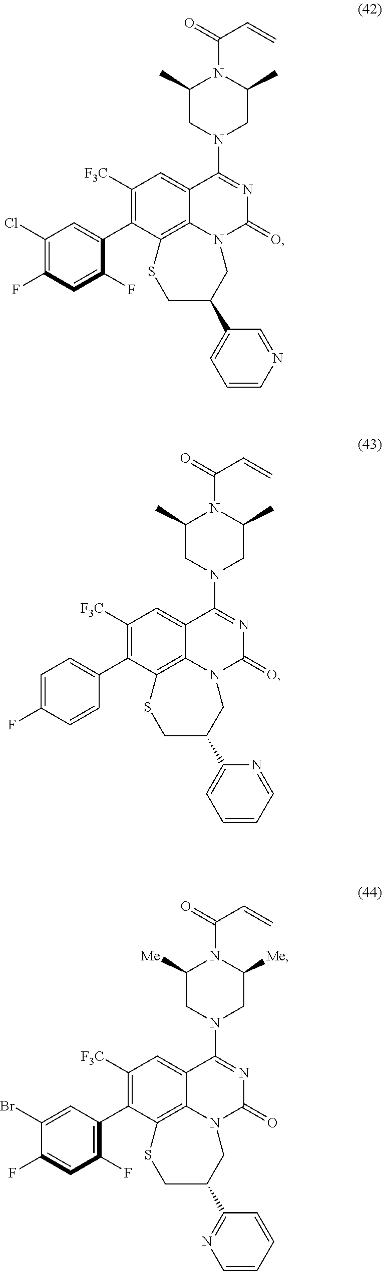 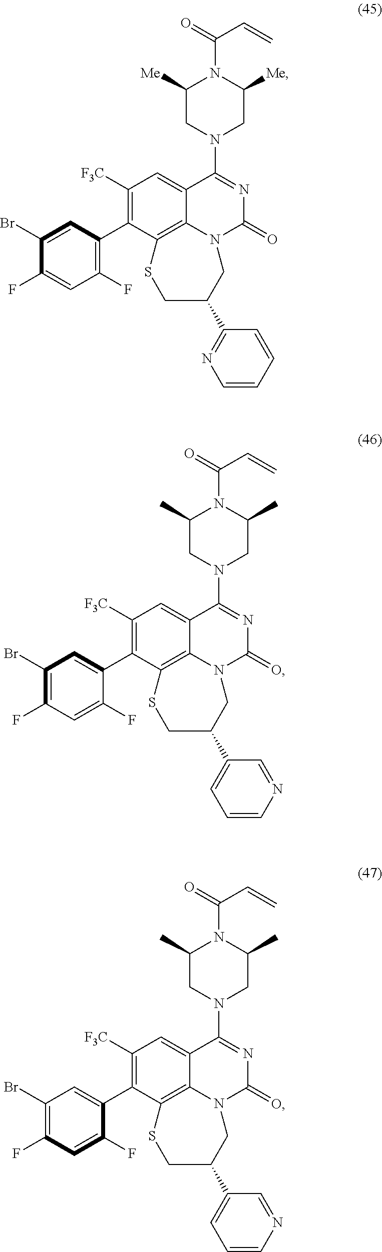 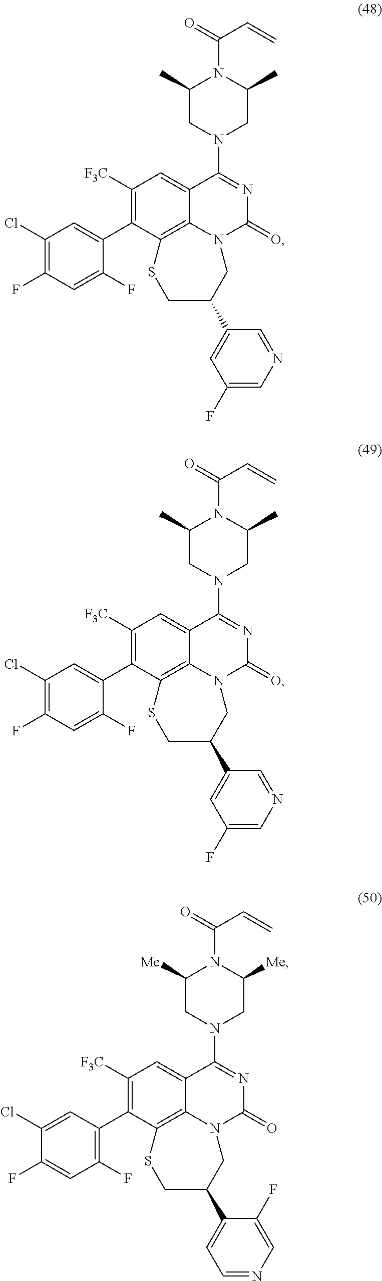 [SMILES: C=CC(=O)N1[C@H](C)CN(c2nc(=O)n3c4c(c(-c5cc(Cl)c(F)cc5F)c(C(F)(F)F)cc24)SC[C@@H](c2cncc(F)c2)C3)C[C@@H]1C ; C=CC(=O)N1[C@H](C)CN(c2nc(=O)n3c4c(c(-c5cc(Cl)c(F)cc5F)c(C(F)(F)F)cc24)SC[C@H](c2cncc(F)c2)C3)C[C@@H]1C ; C=CC(=O)N1[C@H](C)CN(c2nc(=O)n3c4c(c(-c5cc(Cl)c(F)cc5F)c(C(F)(F)F)cc24)SC[C@H](c2ccncc2F)C3)C[C@@H]1C] 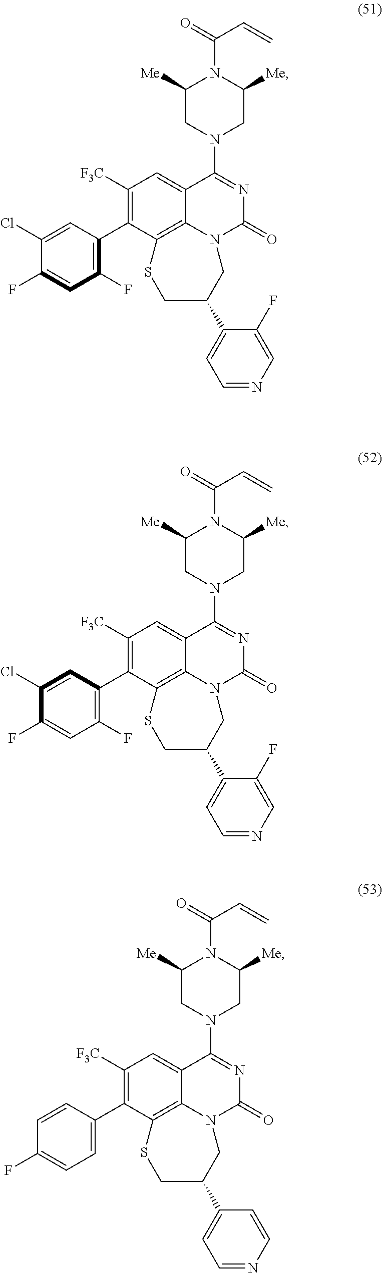 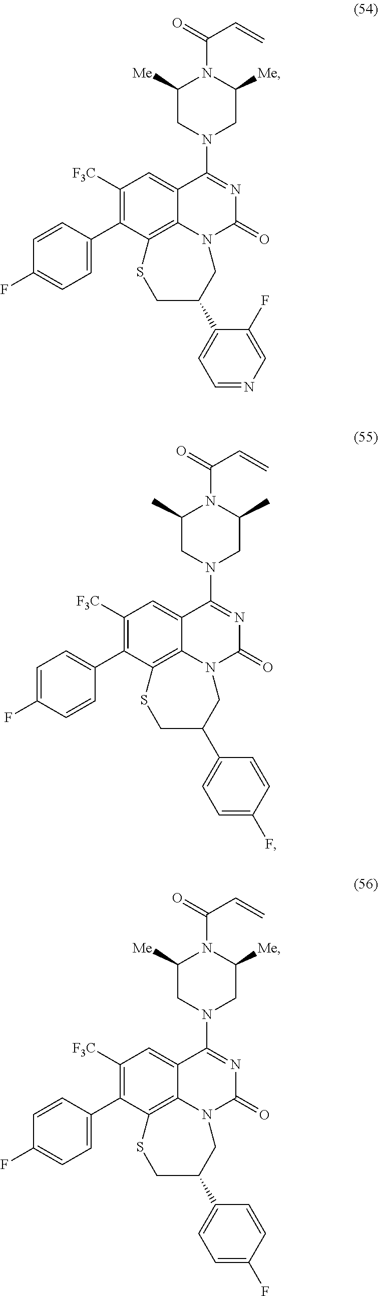 [SMILES: C=CC(=O)N1[C@H](C)CN(c2nc(=O)n3c4c(c(-c5ccc(F)cc5)c(C(F)(F)F)cc24)SC[C@@H](c2ccncc2F)C3)C[C@@H]1C ; C=CC(=O)N1[C@H](C)CN(c2nc(=O)n3c4c(c(-c5ccc(F)cc5)c(C(F)(F)F)cc24)SCC(c2ccc(F)cc2)C3)C[C@@H]1C ; C=CC(=O)N1[C@H](C)CN(c2nc(=O)n3c4c(c(-c5ccc(F)cc5)c(C(F)(F)F)cc24)SC[C@@H](c2ccc(F)cc2)C3)C[C@@H]1C] 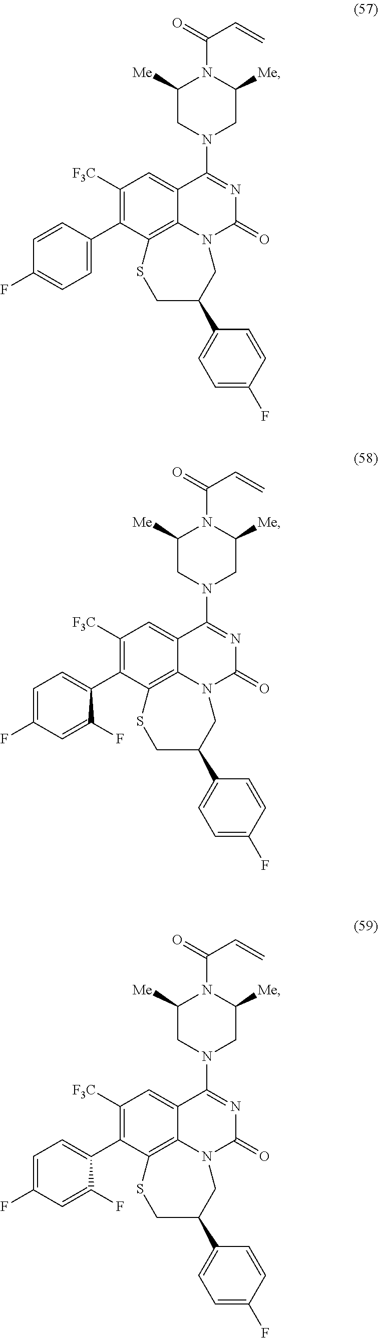 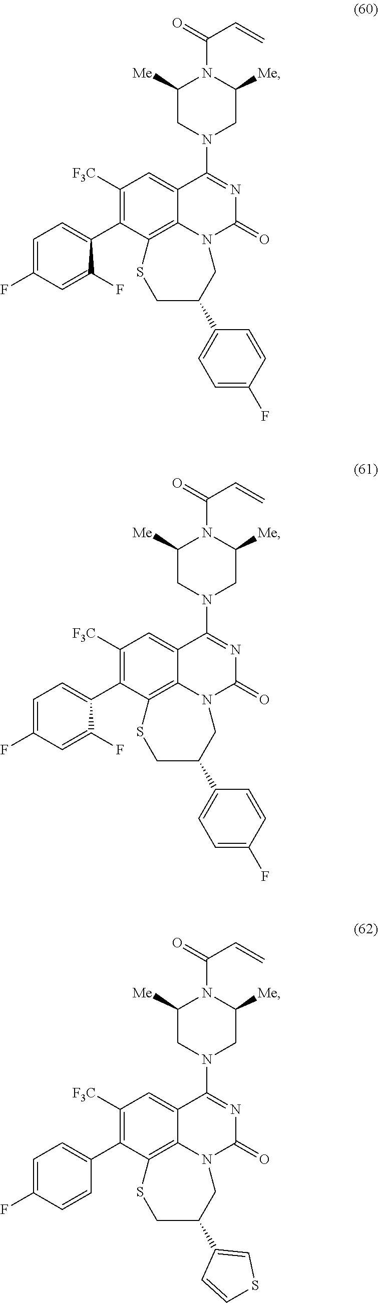 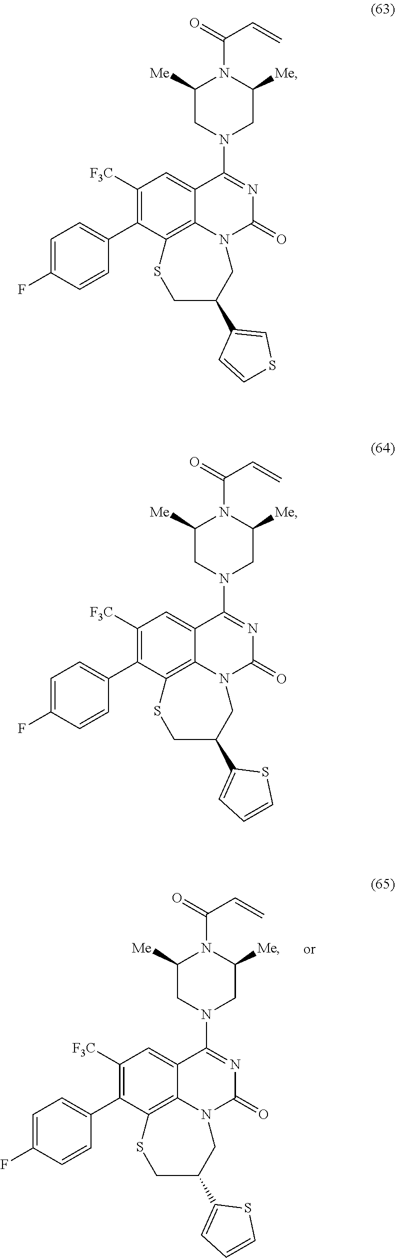 [SMILES: C=CC(=O)N1[C@H](C)CN(c2nc(=O)n3c4c(c(-c5ccc(F)cc5)c(C(F)(F)F)cc24)SC[C@H](c2ccsc2)C3)C[C@@H]1C ; C=CC(=O)N1[C@H](C)CN(c2nc(=O)n3c4c(c(-c5ccc(F)cc5)c(C(F)(F)F)cc24)SC[C@H](c2cccs2)C3)C[C@@H]1C ; C=CC(=O)N1[C@H](C)CN(c2nc(=O)n3c4c(c(-c5ccc(F)cc5)c(C(F)(F)F)cc24)SC[C@@H](c2cccs2)C3)C[C@@H]1C] 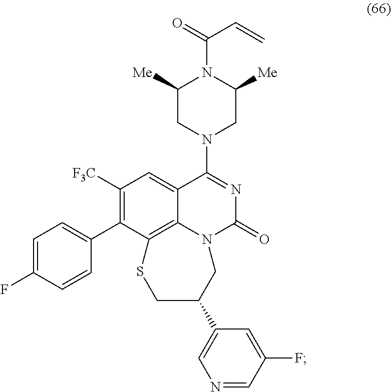 [SMILES: C=CC(=O)N1[C@H](C)CN(c2nc(=O)n3c4c(c(-c5ccc(F)cc5)c(C(F)(F)F)cc24)SC[C@@H](c2cncc(F)c2)C3)C[C@@H]1C]
or a pharmaceutically acceptable salt or solvate thereof.

5. The pharmaceutical combination according to any one of claims 1 to 4, wherein the combination is for simultaneous, sequential, or separate administration.

6. The pharmaceutical combination according to any one of claims 1-5, wherein the combination is a fixed combination.

7. The pharmaceutical combination according to any one of claims 1-5, wherein the combination is a non-fixed combination.

8. A pharmaceutical composition comprising the pharmaceutical combination according to any one of claims 1 to 7 and at least one pharmaceutically acceptable carrier.

9. The pharmaceutical combination according to any one of claims 1 to 7, or the pharmaceutical composition according to claim 8 for use in the treatment of a cancer.

10. The pharmaceutical combination or the pharmaceutical composition for use according to claim 9, wherein the cancer expresses a NF-1 loss of function mutation, wherein the cancer expresses a MAPK mutation or wherein the cancer is N-RAS mutant, H-RAS mutant or K-RAS mutant, or combination thereof.

11. The pharmaceutical combination or the pharmaceutical composition for use according to claim 9 or 10, wherein the cancer comprises a mutation at Q61 selected from Q61R, Q61L, and Q61M.

12. The pharmaceutical combination or the pharmaceutical composition for use or the pharmaceutical composition for use according to any one of claims 9 to 11, wherein the cancer is non-small cell lung cancer (NSCLC), colorectal cancer (CRC), or pancreatic ductal adenocarcinoma (PDAC).

13. The pharmaceutical combination or the pharmaceutical composition for use or the pharmaceutical composition for use according to any one of claims 9 to 11, wherein the cancer is colorectal cancer (CRC).

14. The pharmaceutical combination or the pharmaceutical composition for use or the pharmaceutical composition for use according to any one of claims 9 to 11, wherein the cancer is pancreatic ductal adenocarcinoma (PDAC).

15. The pharmaceutical combination or the pharmaceutical composition for use or the pharmaceutical composition for use according to any one of claims 9 to 11, wherein the cancer is non-small cell lung cancer (NSCLC).

16. The pharmaceutical combination or the pharmaceutical composition for use or the pharmaceutical composition for use according to any one of claims 9 to 15, wherein the cancer is characterized by a mutation in BRAF, NRAS, KRAS, NRAS, or NF-1 or combinations thereof.

17. The pharmaceutical combination or the pharmaceutical composition for use according to any one of claims 9 to 11, wherein the cancer is selected from the group consisting of KRAS-mutant NSCLC (non-small cell lung cancer), KRAS-mutant colorectal cancer (CRC), and KRAS-mutant pancreatic cancer KRAS-mutant pancreatic ductal adenocarcinoma (PDAC).

18. The pharmaceutical combination or the pharmaceutical composition for use according to any one of claims 9 to 17, wherein the combination or composition further comprises an anti-PD-1, anti-PD-L1, or anti-EGFR antibody.

19. A method of treating cancer expressing a MAPK mutation or wherein the cancer is N-RAS mutant, H-RAS mutant or K-RAS mutant, or combination thereof, the method comprising administering the pharmaceutical combination according to any one of claims 1 to 7, or the pharmaceutical composition according to claim 8.

20. The method of claim 19, wherein the cancer is non-small cell lung cancer (NSCLC), colorectal cancer (CRC), or pancreatic ductal adenocarcinoma (PDAC).

21. The method of claim 19, wherein the cancer is non-small cell lung cancer (NSCLC).

22. The method of claim 19, wherein the cancer is colorectal cancer (CRC).

23. The method of claim 19, wherein the cancer is selected from the group consisting of KRAS-mutant NSCLC (non-small cell lung cancer), KRAS-mutant colorectal cancer (CRC), and KRAS-mutant pancreatic cancer KRAS-mutant pancreatic ductal adenocarcinoma (PDAC).

24. The method of any one of claims 19 to 23, wherein the cancer comprises a mutation at Q61 selected from Q61R, Q61L, and Q61M.

25. The method of any one of claims 19 to 24, wherein the pharmaceutical combination or the pharmaceutical composition further comprises an anti-PD-1, anti-PD-L1, or anti-EGFR antibody.
